(19) United States
(12) Patent Application Publication
(10) Pub. No.: US 2024/0000054 A1
(43) Pub. Date: Jan. 4, 2024

(54) CONTROL SYSTEM AND METHODS FOR INSECT BREEDING APPARATUS

(71) Applicant: ENTOCYCLE LTD., London (GB)

(72) Inventors: Paul Samuel HILLMANN, London (GB); Suleman Mohamed HANDULEH, London (GB)

(21) Appl. No.: 18/254,558

(22) PCT Filed: Nov. 25, 2021
(86) PCT No.: PCT/GB2021/053063

§ 371 Date: May 25, 2023

(30) Foreign Application Priority Data
Nov. 25, 2020 (GB) 2018555.9

(51) Int. Cl. A01K 67/033; A01K 29/00

(52) U.S. Cl. CPC A01K 67/033; A01K 29/005

(57) ABSTRACT

A system for controlling a fly breeding apparatus, wherein the fly breeding apparatus comprises one or more enclosures for the containment of a population of flies; wherein the system comprises: one or more input devices, wherein at least one of the one or more input devices is a machine vision system or camera that is configured for imaging the population of flies, or a portion thereof, within at least one of the one or more enclosures; one or more output devices; and a control system; wherein, the one or more input devices, the one or more output devices and the control system are connected to enable the system to control and/or maintain at least one property of a status of the population of flies within the fly breeding apparatus. Methods for controlling a fly breeding apparatus and methods and apparatus for counting flies are also disclosed.

### FIELD OF THE INVENTION

The present disclosure relates to a system and methods for controlling an insect breeding apparatus, in particular a system and methods for controlling and/or maintaining at least one property of the status of a fly population within an insect breeding apparatus.

### BACKGROUND

Insects have been relied on as a source of food for millennia. Insects provide a valuable source of protein, fibre and are also a useful source of many vitamins and minerals. Over recent years there has been growing interest in the field of breeding insects for human and animal consumption. The intentional cultivation of insects, sometimes referred to as ‘insect farming’, has been suggested as one promising way to provide future food security for the ever-increasing population of the world.

Insects have been endorsed by the Food and Agriculture Organisation of the UN (FAO) for their sustainability benefits. Insects can convert plant material to food approximately 10-fold more efficiently than traditionally reared food-producing animals such as pigs and cows. Insects also require far less land and water to sustain growth. Breeding insects has an energy input to protein output ratio of around 4:1 whereas traditional raised livestock has a ratio of 54:1.

Despite the clear advantages of the use of insects as a food source it has historically formed only a small part of the food intake of humans and animals in most countries, particularly in developed countries. While this is partly due to cultural reluctance to change to food from insect sources, this is also largely due to the difficulties and limited understanding of how to farm insects on an industrial scale. While each insect is different, and has differing environmental and nutritional requirements, for the major food producing insects these are becoming understood. What remains a challenge for the industry is how to develop robust, reproducible breeding routines with no or at least minimal manual operator input that are scalable for use on an industrial scale.

Dipteran insects, more commonly known as ‘flies’ are particularly useful in insect farming due to their rapid lifecycle. The Black Soldier Fly (BSF), or Hermetia illucens in particular is known in the art as being efficient at digesting waste organic material and converting this, as part of its growth, into protein and other nutrients suitable for consumption by animals, including humans.

In common with many processes, one of the main challenges that remains with scaling of insect production is consistency. Flies are living creatures and are sensitive to environmental and wider population conditions. Flies can suffer from disease which can have an impact on their breeding ability. Furthermore, optimum breeding occurs only when the flies are healthy, have an environment where they may adopt normal behaviours, and there is an appropriate balance of male and female files. Add to this the fact that inputs such as the type of feed can have a dramatic influence of the health and productivity of a captive insect population, and it is apparent then a means of achieving a consistent population level and health of an insect population within a breeding apparatus becomes of key importance.

To do so, however, is not straightforward. Monitoring the myriad inputs, and environmental and population conditions within a fly breeding apparatus is complex, time-consuming and potentially labour-intensive. This leads to additional costs and may restrict the ability of insect-breeding apparatus to be installed locally on farms, or remotely in rural areas. Some form of reliable monitoring and automation of response would be highly desirable to address these important issues.

The Applicant has already shown that it is possible to breed flies and harvest their larvae in a modular system which is scalable to industrial volumes (WO 2019/053456 A1). While the apparatus described offers a robust, flexible and efficient solution to breeding flies at scale, the control of the fly population at optimum or desired levels is largely through manual intervention requiring operators at site level to monitor conditions within the apparatus and intervene to make any required modifications. Furthermore, there is no means by which to count active fly numbers or monitor the health, behaviour or sex distribution of the fly population.

WO 2019/053439 A2 discusses a waste management system which makes use of larvae to process input waste material. The system comprises a waste management module configured to receive organic waste and to convert the organic waste into a feed for insect larvae and at least one rearing module configured to handle a plurality of trays for holding or housing larvae and to provide the feed to the trays. Some level of automation is described in relation to the waste management module however, no control or automation is applied to controlling and/or optimising the fly population within the system.

There exists a pressing need therefore for a control system for insect, in particular fly, breeding systems that can maintain a healthy fly population with minimum user input. It is an aim of the present invention to address one or more of the disadvantages associated with the prior art.

### SUMMARY OF THE INVENTION

Generally, the invention provides a system for controlling a fly breeding apparatus, wherein the system comprises:
one or more input devices
one or more output devices; and
a control system,
   wherein, the one or more input devices, the one or more output devices and the control system are connected to enable the system to control and/or maintain at least one property of a status of the population of flies within the fly breeding apparatus.

In a first aspect, the invention provides a system for controlling a fly breeding apparatus, wherein the fly breeding apparatus comprises one or more enclosures for the containment of a population of flies:
wherein the system comprises:
one or more input devices, wherein at least one of the one or more input devices is a machine vision system or a camera that is configured for imaging the population of flies, or a portion thereof, within at least one of the one or more enclosures
one or more output devices; and
a control system,
   wherein, the one or more input devices, the one or more output devices and the control system are connected to enable the system to control and/or maintain at least one property of a status of the population of flies within the fly breeding apparatus.

In embodiments, the at least one property of a status of the population of flies is selected from the group consisting of: a total number of flies in the population of flies; a total number of female flies in the population of flies; a total number of male flies in the population of flies; a ratio of the number of female flies to male flies in the population of flies; the health of the population of flies; the behaviour of the population of flies; and combinations thereof.

In embodiments, the one or more inputs are selected from the group consisting of: a further machine vision system or camera; a hyperspectral camera; a gas sensor; a temperature sensor; a pH sensor; a humidity sensor; a weight sensor; a feedback sensor; and combinations thereof.

As used herein, the term ‘feedback sensor’ refers to a sensor that is able to monitor and report back the status of the one or more input devices and/or the one or more output devices, or control system.

In embodiments, the one or more output devices are selected from the group consisting of: lights; feed input controls; larvae input controls; humidifiers; dehumidifiers; heaters; refrigeration or cooling means; gas control valves; mass gas flow devices, such as a fan; motors in automated guided vehicles, suitably motors configured to control the movement of said automated guided vehicles.

In embodiments, the one or more output devices are able to control one or more condition within the fly breeding apparatus, selected from the group consisting of: lighting within the fly breeding apparatus or parts thereof; amount of feed input; moisture content of feed input; nutritional constitution of feed input; frequency of feeding; density of larvae in the feed; humidity; temperature; gas concentration; airflow through the breeding apparatus or parts thereof; and control of automated guided vehicles within the fly breeding apparatus.

In embodiments, the control system is configured to:
a) receive one or more inputs, suitably as data, from the or each of the one or more of the input devices;
b) evaluate the inputs; and
c) send one or more outputs, suitably as instructions, to the or each of the one or more output devices.

In embodiments, the control system is an autonomous optimisation mechanism utilising machine learning. Suitably, the control system comprises one of more machine learning techniques selected from the group consisting of: a neural network; machine learning models; or a combination thereof.

In embodiments, the system further comprises one or more interfaces between the control system and a wired and/or a wireless network for transmitting signals to and/or receiving signals from a local or a remote location. Suitably, the control system is configured to transmit data to and/or receive data from a remote location.

Generally, the invention provides a network, suitably an Internet of Things network, for controlling a fly breeding apparatus, wherein the network comprises:
one or more input devices,
one or more output devices; and
a control system,
   wherein, the network controls and/or maintains at least one property of a status of a population of flies within the fly breeding apparatus.

In a second aspect, the invention provides a network, suitably an Internet of Things network, of connected devices for controlling a fly breeding apparatus, wherein the fly breeding apparatus comprises one or more enclosures for the containment of a population of flies,
wherein the network comprises:
one or more input devices, wherein at least one of the one or more input devices is a machine vision system or camera that is configured for imaging the population of flies, or a portion thereof, within at least one of the one or more enclosures
one or more output devices; and
a control system,
   wherein, the network controls and/or maintains at least one property of a status of a population of flies within the fly breeding apparatus.

In embodiments, the at least one property of a status of the population of flies is selected from the group consisting of: a total number of flies in the population of flies; a total number of female flies in the population of flies; a total number of male flies in the population of flies; a ratio of the number of female flies to male flies in the population of flies; the health of the population of flies; the behaviour of the population of flies; and combinations thereof.

In embodiments, the one or more inputs are selected from the group consisting of: a further machine vision system or a camera; a hyperspectral camera; a gas sensor; a temperature sensor; a pH sensor; a humidity sensor; a weight sensor; a feedback sensor; and combinations thereof.

In embodiments, the one or more output devices are selected from the group consisting of: lights; feed input controls; larvae input controls; humidifiers; dehumidifiers; heaters; refrigeration or cooling means; gas control valves; mass gas flow devices; motors in automated guided vehicles, suitably motors configured to control the movement of said automated guided vehicles.

In embodiments, the one or more output devices are able to control one or more condition within the fly breeding apparatus, selected from the group consisting of: lighting within the fly breeding apparatus or parts thereof; amount of feed input; moisture content of feed input; nutritional constitution of feed input; frequency of feeding; density of larvae in the feed; humidity; temperature; gas concentration; airflow through the breeding apparatus or parts thereof; and control of automated guided vehicles within the fly breeding apparatus.

In embodiments, wherein the control system is configured to:
a) receive one or more inputs, suitably as data, from the or each of the one or more of the input devices;
b) evaluate the inputs; and
c) send one or more outputs, suitably as instructions, to the or each of the one or more output devices.

In embodiments, the network, suitably an Internet of Things network, comprises a wired and/or wireless connection between the one or more input devices, the one or more output devices and the control system.

In embodiments, the network is used in the system of the first aspect of the invention.

Generally, the invention provides a method for controlling a fly breeding apparatus, wherein the method comprises:
a) Providing a fly breeding apparatus;
b) Providing a system for controlling a fly breeding apparatus, wherein the system comprises:
   i. one or more input devices
   ii. one or more output devices; and
   iii. a control system;
c) The control system receives inputs. suitably as data, from the or each of the one or more input devices;
d) The control system evaluates the inputs, for example, data from the or each of the one or more inputs;
e) The control system provides outputs, suitably as instructions, to the one or more output devices;
f) The one or more output devices respond to the instructions to control and/or maintain at least one property of a status of a population of flies within the fly breeding apparatus.

In a third aspect, the invention provides a method for controlling a fly breeding apparatus, wherein the fly breeding apparatus comprises one or more enclosures for the containment of a population of flies;
wherein the method comprises:
a) Providing a fly breeding apparatus;
b) Providing a system for controlling a fly breeding apparatus, wherein the system comprises:
   i. one or more input devices, wherein at least one of the one or more input devices is a machine vision system or camera that is configured for imaging the population of flies, or a portion thereof, within at least one of the one or more enclosures;
   ii. one or more output devices; and
   iii. a control system;
c) The control system receives inputs, suitably as data, from the or each of the one or more input devices;
d) The control system evaluates the inputs, for example, data from the or each of the one or more input devices;
e) The control system provides outputs, suitably as instructions, to the one or more output devices;
f) The one or more output devices respond to the instructions to control and/or maintain at least one property of a status of a population of flies within the fly breeding apparatus.

In embodiments, the at least one property is a total number of flies in the population of flies; a total number of female flies in the population of flies; a total number of male flies in the population of flies; a ratio of the number of female flies to male flies in the population of flies; the health of the population of flies; the behaviour of the population of flies; and combination thereof.

In embodiments, the one or more input devices are selected from the group consisting of: a further machine vision system or a camera; a hyperspectral camera; a gas sensor; a temperature sensor; a pH sensor; a humidity sensor; a weight sensor; a feedback sensor; and combinations thereof.

In embodiments, the one or more output devices are selected from the group consisting of: lights; feed input controls; larvae input controls; humidifiers; dehumidifiers; heaters; refrigeration or cooling means; gas control valves; mass gas flow devices; motors in automated guided vehicles, suitably motors configured to control the movement of said automated guided vehicles.

In embodiments, the one or more output devices are able to control one or more condition within the fly breeding apparatus, selected from the group consisting of: lighting within the fly breeding apparatus or parts thereof; amount of feed input; moisture content of feed input; nutritional constitution of feed input; frequency of feeding; density of larvae in the feed; humidity; temperature; gas concentration; airflow through the breeding apparatus or parts thereof; and control of automated guided vehicles within the fly breeding apparatus.

In embodiments of the first second or third aspect of the invention, the machine vision system comprises at least one camera, suitably the camera is for collecting image data or information. In embodiments, the or each camera has a resolution of greater than 5 megapixels. Suitably, the or each camera has a resolution of 20 megapixels.

In embodiments of the first, second or third aspect of the invention, the machine vision system is configured to image, or images, flies on one of more of: an interior surface of the enclosure or part thereof; an interior volume of the enclosure or part thereof; a plane bisecting the interior volume of the enclosure or part thereof; and combinations thereof.

In embodiments of the first, second or third aspect of the invention, the machine vision system is configured to detect, or detects, the number of flies; the sex of flies; the health status of flies; and/or the behaviour status of flies.

Generally, the invention provides a machine vision system for determining at least one property of a status of a population of flies within a fly breeding apparatus, wherein the machine vision system comprises:
1. a fly breeding enclosure;
2. one or more imaging devices, suitably cameras, aimed inwardly into the interior of the fly breeding chamber.

In a fourth aspect, the invention provides a machine vision system for determining at least one property of a status of a population of flies within a fly breeding apparatus, wherein the machine vision system comprises:
1. an enclosure for the containment of a population of flies;
2. one or more image capture devices, suitably cameras, aimed inwardly into the interior of the enclosure.

In embodiments, the system comprises at least one camera, suitably the camera is for collecting image data or information. In embodiments, the or each camera has a resolution of greater than 5 megapixels.

In embodiments, the machine vision system, suitably the cameras of the machine vision system, is configured to image, or images, flies on one of more of: an interior surface of the enclosure or part thereof; an interior volume of the enclosure or part thereof; a plane bisecting the interior volume of the enclosure or part thereof; and combinations thereof.

In embodiments, the machine vision system, suitably the cameras of the machine vision system is configured to detect the number of flies; the sex of flies; the health status of flies; and/or the behaviour status of flies.

In a fifth aspect, the invention provides a method of counting flies using the system of the first aspect, the network of the second aspect or the machine vision system of the fourth aspect of the invention or the system of the first aspect of the invention.

In a sixth aspect, the invention provides a method of determining the ratio of male and female flies using the system of the first aspect, the network of the second aspect or the machine vision system of the fourth aspect of the invention or the system of the first aspect of the invention.

In a seventh aspect, the invention provides a method of determining the health status of flies using the system of the first aspect, the network of the second aspect or the machine vision system of the fourth aspect of the invention or the system of the first aspect of the invention.

In an eighth aspect, the invention provides a method of determining the behaviour status of flies using the system of the first aspect, the network of the second aspect or the machine vision system of the fourth aspect of the invention or the system of the first aspect of the invention.

In embodiments of the fifth, sixth, seventh or eighth aspect, the method is based on extrapolation of a result from a sample area or volume, wherein the sample area or volume is less than or smaller than the area or volume of the whole area or volume, or a defined part thereof. In embodiments, extrapolation is based on applying a multiplier to the result from sample area or volume based on of the ratio of the sample area or volume to the whole area or volume. Suitably, the multiplier is a simple or weighted multiplier. Suitably, the weighting of the weighted amplifier is based on the anticipated or known variations in fly numbers on different surfaces on volumes compared with the sample area or volume imaged.

In a ninth aspect, the invention provides a fly breeding apparatus comprising the system of any one of the first aspect of the invention, the network of the second aspect of the invention and/or the machine vision system of the fourth aspect of the invention.

### BRIEF DESCRIPTION OF THE DRAWINGS

One or more embodiments of the invention will now be described, by way of example only, with reference to the accompanying drawings, in which:

FIG. 1 shows a schematic representation of an embodiment of a fly breeding apparatus, as described in WO2019/053456A1, to which a control system in accordance with an embodiment of the present invention may be applied.

FIG. 2 shows a schematic representation of a chamber containing flies, typically the fly breeding chamber (grey rectangular box) in which a number of machine vision systems are installed (black circles). FIGS. 2a to 2d show example machine vision system configurations in accordance with embodiments of the present invention.

FIG. 3 shows a workflow for a machine learning platform that may be used to control the apparatus for breeding flies in accordance with an embodiment of the invention.

FIG. 4 shows the results of a comparison of the automated fly counting of the machine vision system of the present invention compared to manual counting.

### DEFINITIONS

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

For convenience, before further description of the present disclosure, certain terms employed in the specification, and examples are delineated here. These definitions should be read in the light of the remainder of the disclosure and understood as by a person of skill in the art. Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described.

All publications mentioned are incorporated herein by reference.

The articles ‘a’, ‘an’ and ‘the’ are used to refer to one or to more than one (i.e. to at least one) of the grammatical object of the article.

As used herein, the term ‘comprising’ means any of the recited elements are necessarily included and other elements may optionally be included as well. ‘Consisting essentially of’ means any recited elements are necessarily included, elements which would materially affect the basic and novel characteristics of the listed elements are excluded, and other elements may optionally be included. ‘Consisting of’ means that all elements other than those listed are excluded. Embodiments defined by each of these terms are within the scope of this invention.

As used herein, the term ‘oviposit’ or ‘ovipositing’ refers to laying of eggs, in particular by an insect. Female insects tend to have ovipositing tubes through which fertilised eggs are laid.

As used herein, the term ‘gravid female’ refers to a female carrying fertilised eggs.

As used herein, the term ‘pre-pupae’ refers to an intermediate stage of development between the larval stage and the pupae stage. In the stage the exoskeleton of the larvae has begun to harden and darken but the larvae still moves and/or feeds. It is to be understood that there is no strict transition from larvae to pre-pupae to pupae, or indeed, larvae to pupae, and the term pre-pupae may in some circumstances be used interchangeably herein or in the literature with the term larvae, for example late-stage larvae, or pupae, for example early stage pupae, depending on the given stage of development.

As used herein, each of the terms ‘eggs’, ‘larvae’, ‘pre-pupae’, ‘pupae’ and ‘flies’ refers to the bulk of the batch referred to. It will be understood that due to natural variation and mixing of batches of different ages, each batch may include minor proportions of developmental stages before and/or after that of the bulk of the batch, for example, pre-pupae may mean a bulk batch of pre-pupae including minor proportions of larvae and pupae or adult flies.

The term ‘maintaining’ as used herein means the tendency towards a stable, or substantially stable equilibrium (i.e +/−a given percentage from a predetermined, or chosen, target level, for example +/−1%, 2%, 5% 10%, 15% or 20% from a predetermined, or chosen, target level, optionally taking into account, or in addition to, the degree of error in the measurement technique of used), or steady-state, of a given property of the status of the insect population, or of the insect breeding apparatus. In the present context, ‘homeostasis’ may refer to maintaining (as defined above) or achieving a steady-state in a property or condition of the fly population within the fly-breeding apparatus. or of the fly-breeding apparatus itself, when controlled by the system of the present invention.

The term ‘controlling’ or ‘changing’ or ‘modifying’ or ‘modulating’ as used herein means the tendency to change or alter a given property of the status of the insect population, or of the insect breeding apparatus. In the present context, ‘controlling’ may refer to changing, suitably from one steady-state condition to another, or suitably to achieve or maintain a predetermined condition, at least one property or condition of the fly population within the fly-breeding apparatus. or of the fly-breeding apparatus itself, when controlled by the system of the present invention.

As used herein the term ‘property’ when referring to the status of the insect population may be, although not limited to, exact or average (average in this context meaning mean, mode or median as appropriate, suitably a numerical mean figure over a given time period) total fly numbers, exact or average egg numbers, exact or average larvae numbers, exact or average pupae or pre-pupae numbers, sex distribution/ratio/numbers of the male and female insects, and/or health of the insects, and/or behaviour of the insects. Suitably, the insects in this context are dipteran insects, suitably flies, suitably black soldier flies.

As used herein the term ‘property’ when referring to the status of the insect breeding apparatus may be, although not limited to, temperature, humidity, gas level concentrations, airflow physical location, or lighting. Such properties may suitably have a direct effect on at least one property of the status of the insect population within the insect breeding apparatus.

As used herein the term ‘status’ refers to the overall condition or state of the insect population, or subset thereof, within the fly-breeding apparatus, or of the fly-breeding apparatus itself, or part thereof, as measured by one or more properties, as defined above, or other.

A ‘predetermined level’ or ‘predetermined condition’ or ‘predetermined criteria’ is understood to mean previously determined parameters or values which allow for a desired outcome, for example, fly numbers to be steady and/or otherwise optimal. The parameters may be measured by suitable measuring equipment or sensors, such as machine vision systems (cameras and/or visual sensors), temperature sensors, gas sensors, light sensors etc. Typically, the measured parameters are compared against the known or control values and maintained or adjusted accordingly so the predetermined condition can be maintained or achieved. Such a comparison and subsequent adjustment may be made by an operator based on their experience. Manual operator input may be replaced by an automated system that relies on a pre-agreed routine, which may have been generated using machine-learning of prior training outcomes or based on real-time feedback loops which monitor and may further adjust conditions based on the result on a given parameter, such as insect or fly numbers, sex, health and/or behaviour.

As used herein, the term ‘machine vision system’ is understood to mean a camera or scanner, or other light-based (wherein the light is in the visual or non-visual band) or visual monitoring technique capable of detecting a property of a fly population. Suitably, the property detected may be the number of flies, the sex of the flies, the behaviour of the flies and/or the health status of the flies. In embodiments, the machine vision system may rely on known or proprietary blob detection methods which detect regions in an image, suitably a digital image, that differ in properties, such as brightness or colour, compared to surrounding regions. Alternatively, or in combination, the machine vision system may rely on known or proprietary feature or shape detection methods that are used to transform the raw image data into symbolic representations used for recognition of shape or patterns. In one embodiment, the term ‘machine vision system’ may mean a system that includes one or more cameras or scanners capable of detecting the number of flies in a breeding chamber or other enclosure containing flies within a fly breeding apparatus.

As used herein, the term ‘input device’ refers to a sensor or device that monitors at least one condition or status of a system, or part thereof. An input device, in the context of the present invention, may monitor any suitable status or condition of the system, or part thereof. For example, the status or condition may be selected from, but not limited to, temperature, humidity, gas content, air flow, light conditions, such as lighting colour, light intensity, light timing, fly number, fly behaviour, sex of flies, weight, positional information, pH etc. Specific examples of input devices may be selected from, but not limited to a machine vision system or camera; a hyperspectral camera; a gas sensor; a temperature sensor; a pH sensor; a humidity sensor; a weight sensor; a GPS sensor and a feedback sensor, such as a sensor or device that reports the status of an output device as herein defined (or an input device as defined hereon, or the control system); and combinations thereof.

As used herein, the term ‘output device’ refers to any means of controlling or modulating the condition or status of a system. An output device, in the context of the present invention, may control or modulate any suitable status or condition of a system, or part thereof. For example, the status or condition may be selected from, but not limited to, temperature, humidity, gas content, air flow, light conditions, such as lighting colour, light intensity, light timing, fly number, fly behaviour, sex of flies, weight, positional information, pH etc. Specific examples of output devices may be selected from, but not limited to air conditioning units, heaters, coolers, humidifiers, dehumidifiers, gas control inlets or outlets, fans or other air transit devices, lights or shades, machine vision systems or cameras, sluices, motors, actuators, switches, alarms etc,

As used herein, the term ‘input’ refers to data or information that defines a status, result or condition of a device or sensor. Suitably, the term ‘input’ refers to data or information provided by an ‘input device’ as herein defined. An input may be fed into a control system for evaluation and/or processing by the control system. An input may be an analogue or digital signal or data stream transmitted through wired or wireless connections. The analogue or digital signal or data stream by be an electronic, radiofrequency, light (visible, UV, IR for example) or any other means suitable for data transmission.

As used herein, the term ‘output’ refers to data or information that is to be transmitted to and understood by a receiving device and result in a predetermined status, result or condition of the receiving device. Suitably, the term ‘output’ refers to data or information sent or transmitted to, and received by, an ‘output device’ as herein defined. An output may originate from a control system after evaluation and/or processing by the control system. An output may be an analogue or digital signal or data stream transmitted through wired or wireless connections. The analogue or digital signal or data stream may be an electronic, radiofrequency, light (visible, UV, IR for example) or any other means suitable for data transmission.

As used herein, the term ‘network’ refers to a group or system of interconnected parts or devices. Suitably the network comprises input devices as herein defined, output devices as herein defined, and a control system as herein defined that interacts through the receiving of inputs as herein defined, from the input devices, evaluation of those inputs by the control system, and sending outputs as herein defined to the output devices to control or modulate a status or condition of a system or part thereof. The network devices may be connected by traditional ethernet connection. The network may be based on an Internet of Things (IoT) architecture. The system may include interface(s) to wired and/or wireless (e.g. cellular or wireless LAN) network for transmitting signals to and/or receiving signals from a remote location. The network may allow for Device-to-Device (D2D) communication in which is defined as direct communication between two mobile users without traversing the Base Station (BS) or core network. The D2D network may be combined with an IoT network. The network may be based on proprietary system bus connectivity which would allow for network, such as IoT, compatibility and be a cost effective and operationally simple means of networking non-bespoke machinery throughout the facility.

As used herein, the term ‘control system’ means a system capable of receiving one or more inputs from one or more input devices, evaluating those inputs and then coordinating control of one or more output devices via outputs that can affect conditions, or parameters required to maintain or adjust a property of the apparatus at a desired level. Such a property may be homeostasis or optimisation of one or more properties of a fly population, or subset thereof.

### DETAILED DESCRIPTION

The invention generally relates to a system and methods for controlling apparatus for breeding insects, suitably dipteran insects. In particular, the invention relates to control of the conditions within the apparatus to promote a preferred, optimised or predetermined state of an insect population, suitably a fly population. The state of the insect population may be defined by one of more of the number of, or the behaviour of, or the health and/or sex of, eggs, larvae, pre-pupae, pupae, and/or adult flies within the apparatus. Suitably, the control system and methods of the present invention may enable and/or maintain a productive and healthy fly population with minimal or no manual operator input.

In one aspect, the invention provides a system for controlling apparatus for breeding insects, suitably flies. In accordance with the present invention the system generally comprises: one or more input devices that provide data (inputs); one or more output devices for control of the fly breeding apparatus, or part or property thereof; and a control system, wherein the control system receives data (inputs) from the one or more input devices, evaluates the data and then sends appropriate actions (outputs) to the output devices in order to control and/or maintain at least one property of a status of the population of flies, or subset thereof within the apparatus.

The one or more input devices may be any suitable sensor or detector for reporting a state of a given condition in the apparatus, or in one or more parts of the apparatus. In embodiments, the apparatus comprises one or more enclosures for containment of the population of insects, suitably flies, and at least one of the one or more input devices is a machine vision system or a camera that is configured for imaging the population of insects, suitably flies, or a subset or portion thereof, in at least one of the one or more enclosures.

In embodiments, the inputs may provide data relating to:
(1) the status of the apparatus, or part thereof, in terms of environmental conditions, such as temperature, humidity, gas concentration levels; or the inputs may report the status of the fly population, for example, the number of eggs, larvae, pre-pupae, pupae, adult flies in apparatus, or in various areas or sections or modules of the apparatus;
(2) the health of the flies;
(3) the behaviour of the flies; and/or
(4) the sex distribution of the population.

In embodiments, the input devices may be selected from the group consisting of: one or more machine vision systems or cameras; hyperspectral camera; gas sensor; temperature sensor; gas sensor; pH sensor; humidity sensor; or weighing sensor. More generally, the fly breeding apparatus may be controlled by fly counting, fly sexing, fly behavioural analysis and fly health analysis.

The number, or type, of input devices is not limited and there can be presented as many input devices and of as many types as is required to allow for reporting of the status of the apparatus such that a suitable response via the one or more outputs may be selected by the control system.

The one or more output devices may be any suitable control element or device or action that can modulate a state of a given condition or parameter in the apparatus, or in one or more parts thereof.

In embodiments, the output devices may selected from, but is not limited to, the group consisting of:
(1) temperature control elements, such as heating pads or cooling fans;
(2) ventilation apparatus to provide air, or modified air to the apparatus, or parts thereof, or extract internal gases from the apparatus or parts thereof;
(3) lights able to vary light conditions;
(4) the amount or type of food input;
(5) means of harvesting insects at one or more stages of production;
(6) means of harvesting insects at one or more stages of production; and/or
(7) means of culling insects at one or more stages of production, or inhibiting breeding.

The number or type of output devices is not limited and there can be present as many output devices, and of as many types, as is required to allow for control of the status of the apparatus, or the modulation or maintenance of one or more parameters or properties within the apparatus, such that a desired outcome is achieved.

In embodiments, the one or more parameters may be any parameter or condition or property that impacts on the number, health, behaviour or sex distribution of the insect, suitably fly, population. Suitably, such parameters may be, although not limited to, temperature, gas concentrations, food input, number of eggs or insects at a given stage of production.

In embodiments, the desired outcome may be any result of the fly breeding process. The desired outcome may be predetermined, i.e. set prior to commencement of the fly breeding process, or may be managed, i.e. altered or changed during the fly breeding process to accommodate a change in, or to maintain, a desired outcome. The desired outcome is typically related to achieving homeostasis in one or more properties of a status of fly population. Suitably homeostasis of a status of a fly population relates to the number, health, behaviour, sex distribution of the fly population. Suitably, desired outcome of the fly breeding process is optimal for the desired output, which may be for example larvae for protein production.

As will be understood, optimisation of a given output, for example, larval production, is multi-factorial, and will be affected by one or more, typically, multiple, sometimes all, outputs at various stages of the breeding process. The impact on the breeding process of any given change in conditions resulting from control of the one or more outputs at different points in the apparatus, whether or not applied with one or more other changes simultaneously or subsequently, can be subtle and difficult to predict, even with the benefit of a trained operator. There is therefore a need for a control system that can oversee this process.

The control system may be any suitable system that can receive data or inputs from the input devices, evaluate the data and provide actions or outputs to the or each of the one or more output devices in order to modulate or maintain conditions within the apparatus to achieve a desired outcome.

In prior art systems, one or more skilled operators would monitor the conditions of the apparatus and the fly population within and make appropriate changes to the various output devices, or change the food provided in the case of altering feed, to optimise conditions, or achieve another desired outcome.

In embodiments of the control system of the present invention, the control system is suitably an autonomous or automated system that may be operated with no or minimal human operator input.

Automation or autonomous control of fly breeding apparatus, suitably the entire fly breeding apparatus, has many advantages, for example, reduction in labour costs, and accuracy of control. There are also significant advantages through the ability to overcome the need for local control of the apparatus through either autonomous control, and/or remote control through some form of communication network, suitably a mobile communications network, Wi-Fi, broadband, or satellite communications. This is particularly important when the apparatus is intended for use in rural or otherwise remote locations where providing a regular skilled workforce to operate the apparatus would be impractical and/or uneconomical.

In embodiments, the control system may be local to the apparatus, i.e. attached to the apparatus, or in the same location. Suitably, the control system may be remote from the apparatus. Suitably, the control system may be ‘cloud-based’ with the control system software operating from one or more servers located at an appropriate geographic location. Such systems may be automated, and/or accessed by one or more trained operators who have oversight for one or multiple fly breeding apparatus set-ups located anywhere in the world.

In embodiments, the system of the present invention may comprise a suitable computational architecture, such as an Internet of Things (IoT) architecture or a proprietary system bus architecture, that links the various inputs and outputs to the control system.

In embodiments, the control system may comprise an operating system that is programmed to evaluate the incoming data or inputs from the input devices and provide instructions or outputs to the one or more output devices to maintain or achieve a desired condition in insect breeding apparatus, suitably a fly breeding apparatus.

In embodiments, based on the inputs, and programming and/or optionally the machine learning or training of the system, an output decision will be determined that seeks to restore or maintain the fly population in a predetermined state, for example an optimal state for larval protein yield and/or quality.

In embodiments, the control system may display appropriate instructions for an operator to action, or suitably, appropriate instructions or outputs may be sent directly to the one or more output devices of an automated fly breeding apparatus.

In embodiments, the results of the outputs may be monitored and fed back as an input (data) so that the control system may adapt its response to the one or more output devices. Suitably, this feedback loop would comprise some element of machine learning, optionally via a neural network, or other suitable models, for to constantly adapt the output response to achieve the desired outcome and/or to compensate for an unexpected result. This feedback may also be used for further optimisation of the training set of the machine learning technique(s) used.

The control system may be configured to transmit the data obtained from the inputs to a local or a remote location. The control system may be configured to detect data matching a predetermined parameter or level and/or signal the parameter or other data to a local or remote location. Alternatively, the control system may be configured to automatically correlate received data to a set of one or more predetermined parameters or levels and transmit the parameter or other data to a remote or local location.

The system may include actuators and/or switches and/or control units in the breeding system configured to receive control signals (outputs) from the control system. The system may include interface(s) to wired and/or wireless (e.g. cellular or wireless LAN) network for transmitting signals to and/or receiving signals from a remote location.

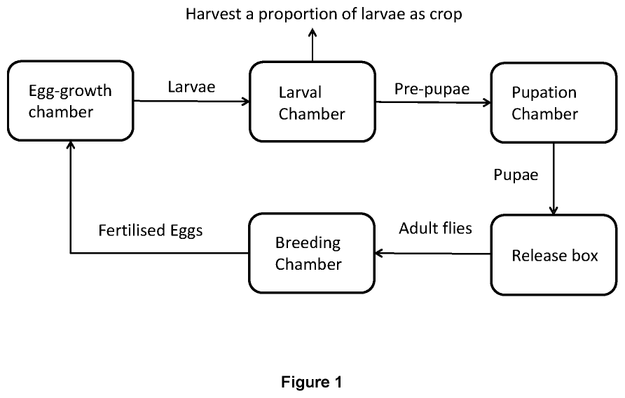

The control system may be used in conjunction with the modular apparatus for breeding flies as described in WO2019/053456A1. FIG. 1 shows a schematic representation of a fly breeding apparatus in accordance with an embodiment of WO2019/053456A1.

Examples of the apparatus described in WO2019/053456A1 comprise five stages or chambers: namely an egg-growth chamber, a larval chamber, a pupation chamber, a release box and a breeding chamber. The egg-growth chamber is where fertilised eggs are incubated to hatch as larvae. The larval chamber is where the larvae grow and mature into pre-pupae. The pupation chamber is where the pre-pupae develop into pupae. The release box is where the pupae emerge as adult flies to be released into the breeding chamber where the adult flies mate and the gravid females oviposit their fertilised eggs which are then returned to the egg-growth chamber. In embodiments, the pupation chamber and the release box may be the same feature, i.e. the same chamber may be where the pre-pupae develop into pupae and where the same pupae emerge as adult flies to be released into the breeding chamber. As is apparent, fertilised eggs laid in the breeding chamber are transferred to the egg-growth chamber to provide a cyclical process. Dealing initially therefore with what is termed herein as the first stage namely the egg-growth, or egg-hatching, stage, it should be noted that the term “first” is merely a suitable label for a starting point on the cycle and not an absolute term in this context.

The invention also provides a system for fly counting, determining the number and/or ratio of male and female flies. Fly behavioural analysis, and/or fly health analysis, the system comprising: at least one machine vision system or camera. The machine vision system or camera may be mounted at an appropriate position for viewing, for example the machine vision system or camera may be mounted to, or view through at least one wall, floor or ceiling of a chamber containing flies, typically the fly breeding chamber. Suitably, the machine vision system or camera may be of suitable resolution to determine the property of the fly or fly population to be monitored.

Input Devices

A number of sensors, status reporting devices and cameras/machine vision systems which may be incorporated into the fly breeding apparatus to obtain data representative of the status or properties or conditions within the apparatus of part thereof in order to maintain and/or achieve a desired outcome, for example homeostasis in a fly population at a predetermined and/or optimal level. While the range of input devices is not limited in number or type, specific exemplary inputs and input devices are discussed in detail below.

Fly Counting

The applicant has previously shown that larvae counting and fly counting can in principle be beneficial to control the quality of larvae and fly batches within the apparatus for breeding flies (see WO 2019/053456 A1). Typically, the flies passing through the outlet of the release box would be counted one by one using for example a proximity sensor such as breaking a light beam or a passive infra-red system. However, counting flies separately as they egress the release box, although possible, becomes challenging on large scale fly population. Furthermore, counting the number of flies that enter the release box does not necessarily reflect the number of healthy flies, able to breed, at a time thereafter.

In accordance with an embodiment of the present invention, an alternative method better suited to large scale fly breeding is counting the number of flies in the breeding chamber, or other suitable cage or receptacle within the breeding apparatus where adult flies are housed, in real-time. In embodiments, taking images of the fly population within the breeding chamber provides one option for counting the number of flies present. Such imaging may be done continuously or intermittently (at regular intervals or at the request of a user or the control system).

In embodiments, the system of the present invention comprises a device or means for fly counting based on imaging data of a surface, plane and/or volume within an enclosure that may contain one or more flies. Suitably, the device for fly counting comprises one or more cameras. Suitably, the device comprises multiple or a plurality of cameras. Suitably, multiple images are taken for each count and the number averaged to improve accuracy. Suitably this average may be a moving average based over a fixed number of counts to improve accuracy in an increasing fly population. Suitably, the device comprises a machine vision system comprising one or more cameras.

The machine vision system, or the one or more cameras of the system, may be mounted to at least one wall, floor or ceiling of a chamber containing flies, typically the fly breeding chamber. Alternatively, or in addition, the machine vision system, or one or more cameras, may be mounted outside of, and viewing into, a chamber containing flies, typically the fly breeding chamber, wherein the walls of the chamber allow the cameras or machine vision system to obtain imaging data from the interior of the chamber. Suitably the surfaces of the breeding chamber allow imaging data to be collected therethrough, for example the surfaces may be perforated (for example, wire, mesh, board with holes through etc.) or are at least sufficiently transparent (for example plastic sheet).

In embodiments, a fly counting device may be used in any chamber or enclosure that is for containment of one or more flies. Suitably the chamber may be selected from one of: an egg-growth chamber; a larval chamber, a pupation chamber; a release box; and a breeding chamber. Suitably, a fly counting system is used in a fly breeding chamber. Alternatively, or in addition, fly counting may occur between defined parts or enclosures within the apparatus, for example between the pupation chamber and the fly breeding chamber.

In embodiments, in addition or instead, a fly counting device may be used for monitoring a population of flies entering, exiting and/or within a cage or room or other enclosure, for example a fly breeding chamber.

In embodiments, the fly counting device may detect the total number of flies present on a given surface or plane, or in a given volume of the fly breeding enclosure. When the counting of flies is a subset of the total number, for example the number of a single surface, or in a plane within the total volume, the total number of flies in the enclosure may be extrapolated from this, for example the extrapolation may based on applying a multiplier (simple or weighted), or other suitable extrapolation technique, to the result from a single or subset of imaged surfaces or planes (or parts thereof) based on of the total number or area of surfaces or the number of planes in the same dimension in total. Such extrapolation may rely on an assumption that the fly numbers in different regions is homogeneous, or there may be a factor or weighting applied to accommodate known or identified variations in fly populations in different areas. Extrapolation of data based on fewer camera inputs allows fewer cameras to be used reducing equipment costs and potentially processing costs in terms of analysing the image data. When multiple cameras or imaging devices are used any regions of a surface or volume to be imaged that overlaps with a surface or volume to be imaged by a second or other camera may be disregarded to avoid double counting of flies. Cameras or imaging devices may be positioned to avoid or minimise such overlap. In addition or alternatively, software techniques may be used to disregard these areas for additional images.

Alternatively, or suitably in addition, the number of flies entering and/or leaving the chamber may also be recorded using the same or secondary machine vision system or one or more cameras, or other techniques, such as breaking of a laser plane. Monitoring the ingress/egress rate of the flies from the chamber is useful to understand the potential rate of change of the population which may be used as separate input data or may be combined with the total fly number data.

If the data on the total number of flies and the ingress and/or egress rate is combined then the difference between the expected number of flies in the chamber based on the number of flies previously present and those since counted as entering may provide information on the state and health of the fly population, for example if there is a discrepancy between these figures it may imply a higher mortality rate than expected, unexpected rearing of flies within the chamber, or potentially a fault with the system.

In embodiments, the device counts flies within the volume of the chamber using the machine vision system. Suitably, this may be achieved by a light source illuminating the chamber and flies within and a machine vision system suitably comprising one or more cameras and/or scanners capable of detecting the light from the one or more flies is reflected back to machine vision system. The source of light may produce light in the visible region, or the non-visible region, such as infra-red or ultraviolet. In embodiments, the source of light may be a laser, suitably a laser that can scan the interior volume of the chamber. Laser light offers consistent light output and acuity that may be advantageous compared to other light sources. In addition, the laser is advantageous for detecting in a plane parallel to the machine vision/camera view, so that the system can detect if flies pass through the laser light acting as a gate.

In embodiments, the laser may be of any suitable form such as semiconductor lasers. The laser may also be a gas laser such as a helium neon gas laser at a wavelength of 543, 594, 612, and 632.8 nm.

In embodiments, the laser may be part of a Gocator™ 2380 sensor system. Gocator™ sensors contain at least one semiconductor laser that emits visible or invisible light and is designated as Class 2M, Class 3R, or Class 3B, the laser may be of relatively low power of at least about 0 mW and at most about 5 mW.

In embodiments, the fly counting system may comprise a laser or light source or a machine vision system only in order to detect the number of flies entering or leaving the fly breeding chamber, or other suitable enclosure where flies are present.

In embodiments, a combination of laser or light source and a machine vision system may be used to provide imaging data from the fly breeding chamber.

In embodiments, any number of cameras or other image acquisition equipment may be used as part of a machine vision system. Suitably, within the fly breeding chamber a machine vision system utilising between 1 and about 16 cameras may be deployed. The cameras may suitably map, or collect image data, from all or one or more parts of the fly chamber. For example, the cameras can map either entirely or across a given sample section of the fly breeding chamber's floor, one or more walls, ceiling and/or space or its interior volume.

The flies may be identified by the machine vision system from their surroundings by any suitable means. Suitably, the machine vision systems of the invention may rely on known blob, feature or shape recognition machine vision methods, or proprietary developments thereof. Suitably, the flies are separated from their surroundings via light spectrum differentiation or using software algorithms to identify flies via their visual characteristics, for example shape, size, movement patterns etc. In embodiments, the identification of flies to enable counting can be done using colour differentiation in monochromatic or multichromatic spectra, size recognition with thresholding to differentiate between singular or multiple flies, via shape recognition per fly or via deep learning algorithms that will learn physical characteristics that identify individual flies.

Once the flies are identified they can be counted in real time and the number of flies in a fly breeding chamber and the rate of flies entering or leaving the fly breeding chamber can be monitored. This can be used to ensure the population within a given volume is kept within a desired range.

In embodiments, the fly breeding chamber may comprise at least 1 camera, suitably at least 2, 3, 4, 5, 6, 7, 8, 9, 10, 11, 12, 13, 14, 15 cameras. The fly cage/room or breeding chamber may comprise at most 20 cameras, typically at most 19, 18, 17, 16, 15, 14, 13, 12, 11, 10, 9, 8, 7, 6, 5, 4, 3 or 2 cameras. Suitably, the fly breeding chamber may comprise 2, 3, 4, 5, 6, 7 or 8 cameras.

In embodiments, the machine vision system captures an image simultaneously from each camera or imaging device present (an image capture event). Suitably, the machine vision system counts the number of flies present from a single image capture. Alternatively, the machine vision system counts the number of flies present from multiple image capture events. Suitably, the number of image capture events per count may be at least 1, 2, 5, 10, 20, 50, 100, 200, 300, 400, 500 or more. Suitably, the number of image capture events per count may be at most 1000, 900, 800, 700, 600, 500, 400 or 300 or less. Suitably, the number of image capture events per count is between 100 and 500, suitably between 200 and 400.

In embodiments, image capture events are separated by a suitable time period to allow for image processing, recordal and/or transmission, to allow the fly population to adjust. Suitably, the time period between image capture events is approximately 0.1 s, 0.2 s, 0.5 s, 1 s, 2 s, 3 s, 4 s, 5 s, 10 s, 15 s or more. This range may reduce as image processing capabilities improve.

In embodiments, the machine vision system or camera may comprise a means for clearing or otherwise maintaining parts of the camera exposed to flies free of obstructions such as settling flies, or other materials that would otherwise disrupt image capture. Suitably, such means may be the lens or lens components, such as the aperture, lens surface or covering, or other parts of the camera essential for obtaining a clear image, for example the autofocus components or the laser source or other lighting. In embodiments, such means may include wiping, for example with a cloth or rubber strip or brush attached to a movable arm, a transparent cover sheet over the camera lens or other affected part of the camera, that may rotated or otherwise periodically moved from in front of the camera to be replaced by a clear cover sheet or part thereof. Suitably, the means is an air curtain or air stream that constantly, periodically and/or intermittently blows air over the camera or affected part at a suitable rate to displace or push any flies from an unwanted position. Suitably the means is a surface coating with low friction that prevents flies from maintaining grip on a surface, which would clear the view with no operating costs. Suitably a low level electrical current, or an increase or decrease in local temperature could be used to discourage flies from landing, this would reduce mechanical part movement. If a plurality of cameras are present, multiple means for clearing flies may be deployed. This embodiment is particularly advantageous as it removes any flies potentially settling on the camera or machine vision system and affecting the accuracy of the counting.

Typically, a lower density of flies produces greater numbers of eggs per female within a given time. A higher density of flies produces a higher amount of eggs per volume of breeding area. At large scale it is necessary to balance these two variables to ensure optimum breeding results.

Further, the optimum population of flies within a given enclosure is dependent on the size of the enclosure, with an experimentally defined optimum density of flies for breeding to be between about 8,000 and about 18,000 flies per cubic metre. For an enclosure of greater than 10 cubic meters an experimentally defined optimum density of flies for breeding is between about 12,000 and about 17,000 flies per cubic metre. The economic, geographic and practical constraints of chamber construction and operator activity mean that an enclosure size is chosen and then a balance between eggs laid per female and total eggs per chamber for a given facility is optimised for and then the density of the fly population is maintained within the desired limits.

In embodiments, the ideal density of flies in the breeding chamber may be at least about 6000 flies/m³, typically at least about 7000, 8000, 9000, 10000 flies/m³ or more. The breeding chamber may comprise at most about 20000 flies/m³, typically at most about 19000, 18000, 17000, 16000, 15000, 14000 flies/m³ or less. Suitably, a desired density of flies in the fly breeding chamber is about 13000 to 18000 flies/m³, most suitably 15000 flies/m³.

Multiple separate populations of flies that have emerged from pupae into flies within a specified time frame which could be from 0 to 72 hours, or from 0 to 48 hours may be released into an enclosure having a fly counting system at the same time. After this, the enclosure may be sealed and the population within complete breeding, die and the enclosure be emptied and reinstated for use. Alternatively, after the defined period, newly emerging flies may be passed to one or more new fly breeding enclosures so that a constant cycle of fly production can be maintained.

In embodiments, the fly breeding chamber, defined as any suitable enclosure in which fly breeding can occur, generally has walls, a ceiling and a floor that can reflect light wavelengths or spectrums, suitably, visible light spectrums, that contrast the bodies of a fly for a given lighting set up. Suitably, as the flies are generally dark in colour, light coloured walls may be used, for example while walls may be used but also light shades of grey, blue, green colours or any other colour depending on the lighting requirements within the chamber.

The shape of the breeding chamber is not limited. In embodiments, the breeding chamber has the shape that may be defined as a regular or irregular cuboid, a rectangular prism, a sphere, a cone, or a cylinder, or any combination of these. Suitably, the breeding chamber has a cuboid or rectangular prism shape for ease of manufacture, although the shape of the breeding chamber may be selected for improved monitoring using the machine vision system of the present invention.

In embodiments, the walls, ceiling and floors of the fly breeding chamber can all be made of the same material or it may be some combination of solid and mesh materials e.g. solid floor and wall but a mesh ceiling.

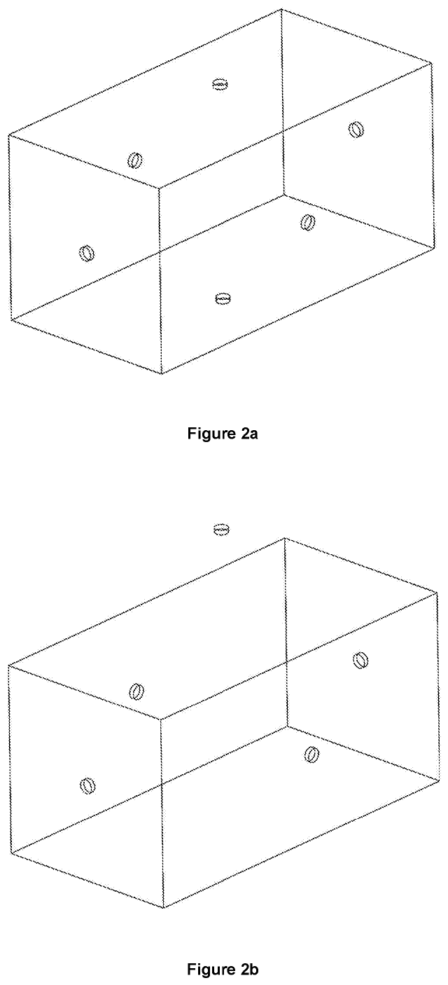

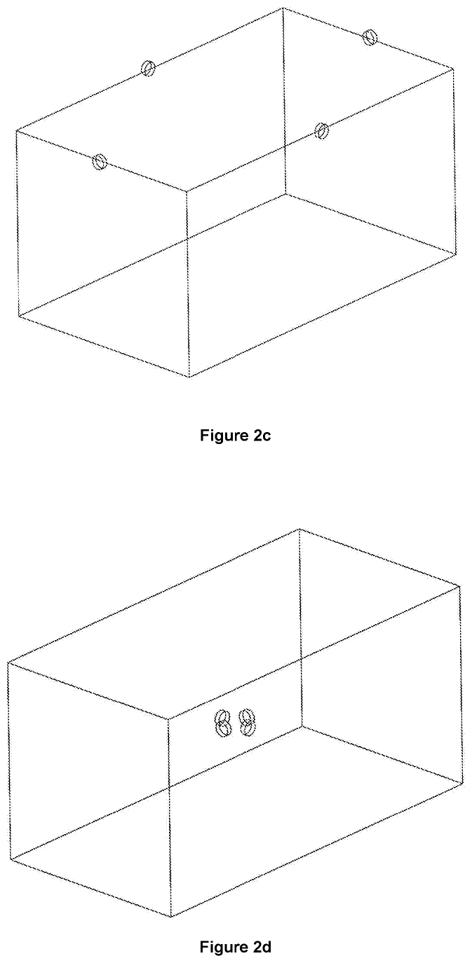

The position of the cameras or other image acquisition equipment in the machine vision system is not limited to any particular arrangement. In embodiments, a group of cameras may be mounted to the walls of the breeding chamber to provide images of the fly population during the release of the flies into the chamber. The mounting of the cameras can be in a position where they are placed, optionally recessed, into the walls, the floor and the ceiling of the chamber and aimed to view the opposing face as the background to the image, as shown in FIG. 2a.

Alternatively, or in addition, one or more cameras may be mounted above an at least partially see-through or transparent ceiling, wall or floor to capture imaging data of an opposing surface. FIG. 2b shows an embodiment where a camera is mounted above an at least partially see-through or transparent ceiling viewing the floor, the ceiling or any horizontal plane within the room by focusing at a specified distance.

Alternate options include cameras in the walls also being able to focus either on the opposing wall or being focused on a point within the room and therefore capturing an image of a vertical plane within the room. Further mounting options can include mounting the cameras on the tops of the walls and viewing down (FIG. 2c) or placing cameras at a location within the chamber and viewing from there (FIG. 2d).

In embodiments, of any of the options camera positioning, the cameras may have variable focal settings to allow a single camera to take images at a specified, and/or different focal distances within the enclosure, thereby allowing a minimum number of cameras to take images within range focus that allow analysis across the entire volume of the chamber as well as on the surfaces within it.

The number of cameras and the resolution of the cameras used is not limited and any suitable combination is encompassed by the invention. Selection of a particular number of cameras or their resolution may depend on the size of chamber used and the imaging data required. In embodiments, this may calculated using the ratio of pixels per fly. The greater the number of pixels per fly means the higher resolution of the image and the more information that can be gathered per image. Suitably, the resolution would be at least 0.25 pixels per mm of the viewed object. Suitably the resolution may be at least 0.5, 1, 1.5, 2, 2.5, 3, 3.5, 4, 4.5, 5, 5.5, 6, 6.5, 7, 7.5, 8, 8.5, 9, 9.5 or 10 pixels per mm. Suitably the resolution may be at most 50, 20, 10, 9.5, 9, 8.5, 8, 7.5, 7, 6.5, 6, 5.5, 5, 4.5, 4, 3.5, or 3 pixels per mm. Suitably the resolution of a given camera, or the overall system would be between 0.5 and 15 pixels per mm. Suitably, 1 to 10 pixels per mm.

For the simplest analysis, the resolution would be at least 1 pixel per mm. For reliable counting, a resolution of at least 2 pixels per mm would be required. This would be equivalent to over 240 pixels per fly. For general analysis on an individual fly, e.g. wings and legs intact approximately 5 pixels per mm would be suitable. For detailed analysis, e.g. discriminating between sexes, identifying behaviours of individual flies or diagnosing pests and pathogens, in embodiments, a resolution of 10 pixels per mm may be required.

All of the above values represent thresholds that allow lesser or greater detail for analysis. In a full system it is likely a combination of cameras providing a range of these values would allow for optimised results.

The resolution of the cameras may be limited due to the higher equipment costs for the system and the longer the processing time of the image. The resolution of the camera may increase as the price of such improved cameras drops in the future. The variation of fly size is limited by biology therefore this is the starting point.

It is estimated that for counting operations the ratio required for resolution per breeding chamber is between 2 to 10 MP/m², suitably 4 MP/m². Therefore, as an example, a 16 MP camera would be able to view an area of 2 m by 2 m. Therefore dependent on required coverage per chamber a number of cameras per square metre of wall space can be calculated and the optimum number of cameras applied to any breeding chamber.

Generally, it is preferred to use pixels required per mm to describe the resolution of the cameras as that can be specified independently of lens type of focal distance.

As an example, for an enclosure of up to about 5 m³ a camera with a resolution of about 12 MP to about 20 MP focused on an opposing wall will provide sufficient pixels per fly to identify and allow the system to count all of the flies on that wall. For operations requiring more detail, such as fly-sexing, this would increase dependent on level of detail required. For these systems pixels per mm is described above. For larger chamber or more complex identification requirements more cameras can be employed to provide a shorter focal length or the resolution per camera increased. A reduced-cost version can also employ a about 5 MP camera with reduced functionality and accuracy.

Typically, at least 1, 2 or 2.4 MP, and at most 3, 4 or 5 MP are required per cubic meter of breeding chamber.

In embodiments, during the release and imaging of the flies the breeding chamber is kept empty of other equipment to ensure that a flat background with minimal shadowing and uniform light levels is provided. In embodiments, the cameras may also be recessed into the walls, suitably behind transparent covers that are able to reflect light similarly to the wall to all other cameras.

An alternate embodiment ensures that the equipment in the breeding chamber is designed to also provide a flat background, this is less effective for counting but reduces labour costs. By way of further explanation, the breeding chamber may comprise other equipment or apparatus such as egg laying substrates and odour attractant mechanisms. The additional apparatus may be in the breeding chamber during firing and could be detected by the machine vision/camera system. In order to reduce confusing image processing, it is desirable design the additional apparatus to look as much like a wall as possible. Alternatively, additional apparatus can be placed into the chamber after fly counting is complete which is more costly.

In an embodiment, the machine vision system can be employed to count using one or more cameras across a given sample area, for example, on a single wall or part thereof, and then that can be used to extrapolate to the fly population across the whole chamber, then for every camera added to the system the amount of extrapolation required is reduced thereby increasing the accuracy of the count and reducing the complexity of the mathematics required to extrapolate to the full population. Alternatively, one or more cameras may be adjusted to have a different viewing angle or focal length to provide multiple images for different areas of the breeding chamber.

The fly population may be counted in real time via the machine vision system and this data may be relayed to the control system manually, for example a readout for an operator to enter, or directly via a computer network, for example a wired or wireless network. Once the fly population of the chamber is approaching the optimum value depending on the flow rate of flies into the chamber the input of the flies may be limited by any suitable means. One example may be by reducing the size of the one or more apertures through which the flies enter. In embodiments, the flies prevented from entry may be diverted to an alternative breeding chamber. Alternatively, or in addition, the apparatus may be controlled by a control system to vary one or more properties to slow the rate of production of flies able to enter the breeding chamber. Alternatively, once the optimum fly population is achieved, earlier stages of fly development, or any flies emerging from pupae may be culled to limit numbers.

In embodiments, the machine vision system or camera may comprise means for clearing and/or wiping the lens (or other essential elements that are required for image capture such as the autofocus apparatus or the laser light or lighting) of the machine vision system or camera. The means for clearing may be selected from the group consisting of: a directed flow of gas such as air, a wiper, a brush, a low friction surface treatment, and/or an electrical or a thermal stimuli. This is particularly advantageous as it removes any flies potentially settling on the lens surface or other component of the machine vision system or camera required for clear image capture and therefore affecting the accuracy of the counting. In embodiments once the population of flies in the breeding chamber reaches an optimum value a signal is sent to an appropriate output on the system and the aperture is closed to prevent further ingress of flies. It is anticipated that this system will always require some degree of extrapolation of the number of the fly population to ensure that the value of flies is correct as it requires that a fly be in the chamber to be counted and therefore a further fly could enter as the aperture is closing. The level of accuracy of this system will be acceptable for almost all cases but where a system requires a higher level of accuracy a counting system that monitors fly ingress through the aperture may be employed.

In an embodiment where the counting takes place during the transit of the flies between the pupation chamber and the fly breeding chamber, a camera or imaging system, for example a 3D laser imaging system, may be mounted parallel to the direction of travel of flies passing from the pupation chamber to the fly breeding chamber. This 3D laser imaging system applies a laser line across a given distance, up to about 1.3 m width in this iteration and anything that passes through volume covered by the line is detected. In this way it can detect all flies that pass through the laser beam and they can be counted. The laser line may also be up to about 1 m, 2 m, 3 m, 4, m, 5, m or 6 m width.

In alternative embodiments, the number of flies may be determined using weighing sensors. Any form of weighing flies in one or more parts of the breeding apparatus is contemplated. In embodiments, a datum or tare mass of either the pupation chamber and/or the fly breeding chamber are measured using a suitable means, such as one or more load cells or weighing scales. Suitable means of weight measurement may be any device capable of measuring weight by either compressive or tensile load. The introduction of flies would then either decrease the mass of the pupation chamber or increase the mass of the fly breeding chamber and this change would be measured. Sampling of the fly population will provide an average mass per fly of the population and then this can be used to extrapolate fly populations within the breeding chamber.

Fly Sexing

To achieve or maintain a desired or optimal breeding activity, and consistent egg yield, within a fly breeding apparatus, as well as the number of flies, the distribution or ratio of male and female flies is also important. The ability to accurately sex and then count the number of male and female flies is therefore important.

As an example, most flies, including Black soldier flies only mate once (Tomberlin J K, Sheppard D C, Joyce J A (2002) Selected life-history traits of black soldier fly (Diptera: Stratiomyidae) reared on three artificial diets. Ann Entomol Soc Am 95:379-386) and therefore an imbalance between the number of males and females directly reduces the number of eggs produced.

Studies have shown that environmental conditions during the development of black soldier flies have an effect on the sex of the flies produced and therefore in order ensure a desired female fly population the sex of the flies must be determined in order to provide the data that will allow optimisation of the ratio of male and female flies in a fly population.

Fly sexing may be performed by a machine vision system that acquires and analyses images across a sample area, sample population or the entire population and may also be used to determine the number of males or females within an enclosure via visual, or otherwise outwardly apparent, sex characteristics.

In embodiments, the system for sexing flies may be the same camera or machine vision system as used for fly counting described above. All features described in respect of fly counting above, and the arrangement or properties of the cameras and apparatus may be equally applicable to fly sexing, unless otherwise further defined below.

A fly sexing system may be used in any one of: egg-growth chamber, pupation chamber, release box and breeding chamber. Preferably a fly sexing system is used in the fly breeding chamber.

The fly breeding room/cage/chamber may comprise at least 1 camera, suitably at least 2, 3, 4, 5, 6, 7, 8, 9, 10, 11, 12, 13, 14, or 15 or more cameras. The fly cage/room or breeding chamber may comprise at most 20 cameras, suitably at most about 19, 18, 17, 16, 15, 14, 13, 12, 11, 10, 9, 8, 7, 6, 5, 4, 3 or less cameras. Suitably, the fly cage/room or breeding chamber may comprise 2 to 4 cameras.

In embodiments, the optimum population of female flies versus male flies within a given enclosure, for example the fly breeding chamber may be between 40:60 to 60:40, suitably 50:50 so that ideally the numbers of female flies and male flies in the breeding chamber are similar or the same.

In embodiments, the desired density of female flies in the breeding chamber may be about 4,000 and about 9,000 female flies per cubic metre. For larger chambers the ideal density of female flies in the breeding chamber may be about 6,000 and about 8,500 female flies per cubic metre.

In embodiments, the desired density of male flies in the breeding chamber may be about 4,000 and about 9,000 male flies per cubic metre. For larger chambers the ideal density of male flies in the breeding chamber may be about 6,000 and about 8,500 male flies per cubic metre.

In embodiments, the desired density of female flies in the fly breeding chamber may be at least about 3,000 flies/m³, typically at least about 3,500 flies/m³, suitably at least about 4,000 flies/m³. The fly cage/room or breeding chamber may comprise at most about 10,000 flies/m³, typically at most about 9500 flies/m³ and suitably at most about 9,000 flies/m³. Suitably, the fly breeding chamber may comprise around 6,500 flies/m³.

In embodiments, the desired density of male flies in the fly breeding chamber may be at least about 3,000 flies/m³, typically at least about 3,500 flies/m³, suitably at least about 4,000 flies/m³. The fly cage/room or breeding chamber may comprise at most about 10,000 flies/m³, typically at most about 9,500 flies/m³ and suitably at most about 9,000 flies/m³. Suitably, the fly breeding chamber may comprise around 6,500 flies/m³

In order to perform fly sexing more easily via machine vision systems the contrast of the imaging data described above is advantageous, i.e., at least one wall of the breeding chamber may be white or coloured in shades of grey, blue, green colours or any other colour depending on the lighting requirements within the breeding chamber. A plurality of walls within the breeding chamber may be coloured.

A group of cameras may be mounted to the walls of the breeding chamber to provide images of the fly sex characteristics e.g. the female ovipositor in order to determine the ratio of male to female flies in the breeding chamber. Other characteristics of fly sex are differences in colour, body size, head shape and size and colours of excretions. A visual difference between a male and female fly is identified, for example, the difference between male and female black soldier flies is visible, allowing someone skilled in the art to determine the sex by identification of the female ovipositor. While this identification cannot be done quickly or reliably by a human meaning it cannot be done on a large population, the fly counter technology described above provides the technological architecture for distinguishing between the male and female populations of the chamber with the following additional requirements.

In principle the same machine vision set up as described for fly counting can be used. For example, the mounting of the cameras can be in a position as shown in FIGS. 2a to 2d.

The number of pixels required to identify a fly can be significantly lower than the number of pixels to identify the sex of a fly, for example to see an ovipositor on a female fly. Consequently, a machine vision system that is intended for fly sexing, either alongside or instead of fly counting, may require a significantly higher resolution camera to provide images of sufficient resolution for determining the sex of the flies.

Suitably, the resolution of the camera (or combined system) would be at least 5 pixels per mm. Suitably the resolution may be at least 6, 7, 8, 9, or 10 pixels per mm. Suitably the resolution may be at most 50, 40, 30, 20, 15, 14, 13, 12, 11 or 10 pixels per mm. Suitably the resolution of a given camera, or the overall system would be between 5 and 15 pixels per mm. Suitably, 8 to 12 pixels per mm.

All of the above values represent thresholds that allow greater detail for analysis. In a full system it is likely a combination of cameras providing a range of these values would allow for optimised results.

The machine vision or camera output informs the control system of the fly breeding facility of the imbalanced sex ratio, this then can increase the number of larvae in the breeding system to make up for the shortfall in egg production that will occur. Environmental, nutritional, hormonal or other factors can be used to affect the sex of the flies enabling the system to be able to identify and counter these if they are naturally occurring or to artificially alter them to optimise the sex ratio of the flies

Furthermore, if the relationship between the sex of the flies in a given area of the enclosure, for example, on the wall, and the sex of the flies in the chamber in total is consistent for varying populations then fewer cameras would be required, relying instead on iterating or extrapolating data from a sample group of flies.

In embodiments, a computer algorithm may be employed to enable the identification of the sex of flies and/or for counting which may be done via shape recognition per fly or via deep learning algorithms that will learn physical characteristics such as behaviour or movement patterns that identify individual flies and their sex. In embodiments, the computer algorithm may make use of machine learning techniques, such as those based on neural networks of other suitable models.

In embodiments, the machine vision system or camera may comprise means for clearing and/or wiping the lens (or other essential elements that are required for image capture such as the autofocus apparatus or the laser light or lighting) of the machine vision system or camera. The means for clearing may be selected from the group consisting of: a directed flow of gas such as air, a wiper, a brush, a low friction surface treatment, and/or an electrical or a thermal stimuli. This is particularly advantageous as it removes any flies potentially settling on the lens surface or other component of the machine vision system or camera required for clear image capture and therefore affecting the accuracy of the sexing.

Fly Behaviour Analysis

A camera system or a machine vision system may also be able to acquire and analyse images of fly behaviour such as movement. In embodiments, such analysis may be conducted individually, or by a sample across the population in an enclosure. In embodiments, the analysis may be by any suitable method including human analysis and/or in an automated fashion using a machine learning algorithm or AI to determine the behaviour it represents and then create feedback systems within the breeding system.

By knowing the behaviour of the flies, continuous optimisation of conditions for breeding can be carried out as part of standard breeding processes. For example, it has been shown that a high density of flies in one area of an enclosure, for example on egg laying substrate, can reduce the total number of eggs laid and increase the chances of the eggs being laid in the wrong place leading to a reduction in the number of eggs per female achievable. In addition, the behaviour of male and female flies in the breeding chamber differs during mating.

If the environment within the enclosure is sub-optimal then the mortality rate of the flies within the enclosure will be higher, resulting in higher numbers of dead flies on the floor of the enclosure and less flies on the walls or flying within the enclosure.

For all of the above cases an understanding of the distribution throughout the breeding chamber is required to be able to take actions to reduce the impact of them. The fly counter technology described above in respect of fly counting and fly sexing can also be adapted to provide the technological architecture for distinguishing the behaviour of flies within the vessel with the following additional requirements.

A system for fly behavioural analysis may be used in any one of: egg-growth chamber, larval chamber, pupation chamber, release box and breeding chamber. Preferably a system for fly behavioural analysis is used in the fly breeding chamber.

In principle the same machine vision set up as described for fly counting and fly sexing can be used. For example, the mounting of the cameras can be in a standard position where they are recessed into the walls, the floor and the ceiling of the chamber and viewing the opposing face as the background to the image, as described above and shown in FIG. 2a to 2d.

As for the fly counting and fly sexing inputs, in order to more easily perform fly behavioural analysis via machine vision systems, at least one wall of the breeding chamber may coloured in white or light shades of grey, blue, green colours or any other colour depending on the lighting requirements within the breeding chamber to contrast with the dark flies. One or more of the walls within the breeding chamber may be coloured. The walls, ceiling and floors of the breeding chamber can all be made of the same material or it may be some combination of solid and mesh materials e.g. solid floor and wall but a mesh ceiling.

One or a group of cameras may be mounted to the walls of the breeding chamber to provide images of the flies in order to monitor, identify and optionally score their behaviour.

In embodiments, for a chamber of up to about 5 m³ either a about 12 MP or about 20 MP camera focused across a wall will provide enough pixels per fly to identify and allow the system to determine the behaviour of all of the flies on that wall. For larger chambers or more complex identification requirements more cameras can be employed or the resolution per camera increased. A cost reduced version can also employ a about 5 MP camera with reduced functionality and accuracy.

Typically at least 1, 2 or 2.4 MP, and at most 3, 4 or 5 MP are required per cubic meter of breeding chamber.

Suitably, the resolution of the camera (or combined system) would be at least 5 pixels per mm. Suitably the resolution may be at least 6, 7, 8, 9, or 10 pixels per mm. Suitably the resolution may be at most 50, 40, 30, 20, or 10 pixels per mm. Suitably the resolution of a given camera, or the overall system would be between 5 and 15 pixels per mm. Suitably, 8 to 12 pixels per mm.

All of the above values represent thresholds that allow lesser or greater detail for analysis. In a full system it is likely a combination of cameras providing a range of these values would allow for optimised results.

Without limitation, and for example only, at least two methodologies may be applied for behavioural analysis of flies.

Firstly, for individual monitoring, one high resolution camera, for example with 10 pixels per mm may be applied, at a high frame rate, such as over 5 frames per second to allow for individual fly behaviour to be analysed. Suitably the frame rate is over 10, 15, 20 or 25 frames per second.

Secondly, for population monitoring, the number of cameras may be dependent on the behaviours being monitored, it is assumed that all areas of interest will require monitoring, so for a standard chamber multiple cameras may be deployed, for example three cameras, each set to a relatively high resolution, for example 2 pixels per mm would be employed, one monitoring a specific area of the chamber, for example, the oviposition substrates, the water application area or the floor of the chamber and one monitoring a sample area of wall. Combinations of these areas will provide sufficient information to understand fly population behaviours.

In order to map the full distribution of flies within the chamber, cameras are required to image one or more interior surfaces of the enclosure, and/or the volume of space within the whole enclosure. The volume within the enclosure should be analysed in minimum one plane, preferably two or three planes to ensure maximum measurement accuracy. The resolution of cameras required to count flies as described hereinabove are typically sufficient to determine the behaviour of flies.

Features added into the breeding chamber, such as water provision, feed provision, laying substrate, odour provision, would all need to be identifiable by the fly behaviour analysis machine so that the behaviour can be accounted for. By way of further explanation, additional equipment or apparatus in the chamber such as a water feeder (and/or egg laying substrate, odour provision units) will change the behaviour of the flies, for example flies might congregate at the water feeder to drink. In embodiments, the behavioural analysis machine will need to identify the location of the additional equipment or apparatus i.e. the water feeder in order to map out the behaviour of the flies in relation to it.

In embodiments, the lighting for the behavioural monitoring system may be different to the fly counting system as it is based around monitoring the behaviour of the flies in breeding lighting conditions rather than the different lighting conditions used for releasing the flies into the chambers. Fly counting and fly behavioural analysis may be performed in the same chamber. For example, the flies may be counted on initial filling of the breeding chamber and behavioural analysis may be performed throughout the whole lifespan of the chamber. Typically, the lighting during the initial filling of the chamber is different to the lighting during the rest of the lifespan of the chamber (attractant lighting on filling versus lighting to attract flies to mate or lay eggs).

In embodiments, the machine vision system or camera may comprise means for clearing and/or wiping the lens (or other essential elements that are required for image capture such as the autofocus apparatus or the laser light or lighting) of the machine vision system or camera. The means for clearing may be selected from the group consisting of: a directed flow of gas such as air, a wiper, a brush, a low friction surface treatment, and/or an electrical or a thermal stimuli. This is particularly advantageous as it removes any flies potentially settling on the lens surface or other component of the machine vision system or camera required for clear image capture and therefore affecting the accuracy of the behavioural analysis.

Fly Health Analysis

Further, a machine vision system may be used to acquire and analyse images of individual flies, or a sample across the population in the breeding chamber which can be analysed to determine the health of an individual or group of flies. Health may be measured by detecting any abnormalities in fly shape or condition or behaviour that can then be used to diagnose any physical problems with the flies, e.g. damaged wings, bacterial infections, that are visible. As appropriate, varying spectrums of light could be used if required to identify specific issues that are not shown within the visible spectrum.

There are two elements to the output of fly health, the first is that healthy flies will produce consistent egg yields, the second is that the healthier the fly the higher the viability of the eggs laid. Ensuring the consistency of these two factors ensures that the number of larvae that hatch in the system is more consistent and/or predictable.

The fly sexing machine technology described above may provide the technological architecture required for analysing the health of the flies in the breeding chamber. Typically, the camera resolution required to identify a female ovipositor would be enough to analyse the majority of visual imperfections in the fly population with the following additional requirements.

In order to perform fly health analysis via machine vision systems more easily, contrasting surfaces may be provided. At least one interior surface of the breeding chamber may be coloured in light shades of grey, blue, green colours or any other colour depending on the lighting requirements within the breeding chamber. A plurality of walls within the breeding chamber may be coloured. The walls, ceiling and floors of the breeding chamber can all be made of the same material, or it may be some combination of solid and mesh materials e.g. solid floor and wall but a mesh ceiling.

A group of cameras may be mounted to the walls of the breeding chamber to provide images of the flies in order to monitor and determine health characteristics.

In principle the same machine vision set up as described for fly sexing can be used. For example, the mounting of the cameras can be in a position, optionally recessed into the walls, the floor and/or the ceiling of the chamber and viewing the opposing face as the background to the image, as shown in FIGS. 2a to 2d.

Suitably, the resolution of the camera (or combined system) would be at least 5 pixels per mm. Suitably the resolution may be at least 6, 7, 8, 9, or 10 pixels per mm. Suitably the resolution may be at most 50, 40, 30, 20, or 10 pixels per mm. Suitably the resolution of a given camera, or the overall system would be between 5 and 15 pixels per mm. Suitably, 8 to 12 pixels per mm.

All of the above values represent thresholds that allow lesser or greater detail for analysis. In a full system it is likely a combination of cameras providing a range of these values would allow for optimised results.

All of the above values represent thresholds that allow lesser or greater detail for analysis. In a full system it is likely a combination of cameras providing a range of these values would allow for optimised results.

The health of the flies may be interpreted by a human operator or may be based on matching parameters to known characteristics of fly disease or disorder. In embodiments, a large data set of images of flies considered healthy is captured and this would then become the standard model for an algorithm to operate from. Any difference from this model would be highlighted to a user and the user would be able to diagnose remotely if this is an issue, which would then update the algorithm and a database of issues would be automatically diagnosed from that point onwards.

Examples of fly health issues and related characteristics that can be diagnosed using the above machine vision system include but are not limited to:
Undersized flies—indicating feed or environmental issues with the larvae breeding systems.
Damaged wings—indicating issues with the release mechanisms and chamber between pupation and the breeding chamber.
Bacterial or fungal infections—Indicating feed or environmental issues with the larvae breeding systems.
Presence of white (or other coloured) mites or other pest species—indicating issues with the pupae storage system.

In further embodiments of the invention non-visible spectrum lighting and corresponding camera sensors may be used to identify fly health that is not visible to the human eye. Thus, extending diagnostic criteria beyond any previously employed.

In embodiments, the machine vision system or camera may comprise means for clearing and/or wiping the lens (or other essential elements that are required for image capture such as the autofocus apparatus or the laser light or lighting) of the machine vision system or camera. The means for clearing may be selected from the group consisting of: a directed flow of gas such as air, a wiper, a brush, a low friction surface treatment, or an electrical or a thermal stimuli. This is particularly advantageous as it removes any flies potentially settling on the lens surface or other component of the machine vision system or camera required for clear image capture and therefore affecting the accuracy of the health analysis.

Further Sensors

A variety of sensors may be deployed instead of, or suitably in conjunction with, the machine vision systems described above.

The sensors may be deployed in any one of: egg-growth chamber, larval chamber, pupation chamber, release box and breeding chamber, or part of each thereof. Monitoring of temperature and humidity has been described at all stages of the fly breeding cycle within the apparatus of WO2019/053456A1, and sensors may be deployed for this purpose in all of the egg-growth chamber, the larval chamber, the pupation chamber, the release box and the breeding chamber. Suitably, sensors may be deployed to detect the temperature and/or humidity at multiple points in each area, such as above each rack.

In embodiments, at least one temperature sensor may be deployed in the breeding chamber in order to obtain environmental data within the breeding chamber. A plurality of temperature sensors may be deployed. A temperature sensor as referred to herein is an electronic device that measures the temperature of its environment and converts the input data into electronic data to record, monitor, or signal temperature changes. The at least one temperature sensor may be an infrared (R) temperature sensor or a thermal camera. Typically, the at least one temperature sensor may be selected from the group consisting of: a negative Temperature Coefficient (NTC) thermistor, a Resistance Temperature Detector (RTD), a Thermocouple, or a Semiconductor-based sensor.

The environment in the breeding chamber and the release box is carefully controlled. The temperature in the release box may be at least about −4° C. and at most about 28° C. The temperature in the breeding chamber may be at least about −2° C. and at most about 28° C.

The environment in the breeding chamber is carefully controlled. The temperature is generally maintained in the release box to be above 200° C. More suitably the temperature is generally maintained in the release box to be above 25° C. Suitably the temperature within the release box is above 21° C., 22° C., 23° C., 24° C., 25° C., 26° C., 27° C. or 28° C. The temperature is generally maintained in the release box to be below 40° C. More suitably the temperature is generally maintained in the release box to be below 35° C. Suitably the temperature within the release box is no more than 29° C., 30° C., 31° C., 32° C., 33° C., 340° C., 35° C., 36° C., 37° C., 38° C., 39° C. or 40° C. Suitably the temperature is within the range of from 23° C. to 35° C. Suitably, the temperature is within the range of from 25° C. to 32° C. More suitably, the temperature is within the range of from 26° C. to 30° C.

Alternatively, or in addition, temperature sensors may be deployed in or near one or more of the palletised stacks of containers can be used to store larvae during their growth period i.e. in the larval chamber. If the environmental and biological conditions within the pallets are within acceptable bounds then the larvae will remain within the stacks as all of their needs are met. If there is a local variation in temperature outside of the bounds acceptable for the larvae then the larvae will attempt to escape from the stack. Mounting temperature sensors, such as infra-red detecting cameras around the stacks, either in the ceiling or on the walls of the chamber housing the larvae allowing optimum viewing angles means that the temperature of the stacks of containers can be monitored to detect either local hot or cool spots that may lead to larval discomfort, or larvae that have already escaped meaning that there is an issue.

This system can be combined or used independently with Automated Guided Vehicles to provide closer inspection of the floors and the stacks from floor level. The combination of these will allow for detection of issues via thermal properties and extrapolation of larvae wellbeing based on activity, which can be measured via thermal measurement.

At least one humidity sensor may be deployed in the breeding chamber in order to obtain environmental data within the breeding chamber. A plurality of humidity sensors may be deployed. A humidity sensor (or hygrometer or psychrometer) senses, measures and reports moisture or relative humidity and optionally air temperature. Typically, at least one humidity sensor, suitably at least one capacitive humidity sensor is used. For example, Frass moisture content after pupae separation may be measured (sample analysis). Advantageously, the frass moisture content provides information about how much processing the frass needs to undergo before sale, the effectiveness of the upstream climate control systems and about optimisation of the feedstock inputs. Further, the moisture content of breeding substrate overtime may be measured (in line sensors and/or sample analysis).

The humidity within the breeding chamber is carefully controlled. The relative humidity in the breeding chamber is generally held below 75% relative humidity (RH) as measured by a psychrometer or a hygrometer. Suitably the relative humidity in the breeding chamber is at least 10%. Suitably the relative humidity is above 20%. The relative humidity may be above 25%, 30%, 35%, 40%, 50%, or 55%. The relative humidity may be a most 70%. The relative humidity may be at most 60%, 55%, 50%, 45%, 40%, 35% or 30%. Suitably the relative humidity may be in the range from 10% to 80%. More suitably the relative humidity may be in the range of from 20% to 70%. The relative humidity in the breeding chamber may be at least about 65% and at most about 75% and is suitably 70%.

It may be advantageous to measure the temperature and the humidity of the breeding chamber as a whole, and/or the breeding area and/or in or around on or more of the breeding containers or shelves individually or simultaneously to obtain breeding chamber climate data. The temperature and humidity may be measured at the same time or sequentially.

Suitably, the moisture content of the feed input is measured, typically by in line sensors, in order to account for climate changes in the breeding chamber due to the feed source.

Combinations of moisture content of feed and humidity within the chamber can be used to determine necessary refresh rates of air, mechanical changes to bioconversion equipment or harvest timings for the larvae within the breeding containers or shelves.

At least one hyperspectral camera or hyperspectral image sensor may be deployed to determine the nutritional characteristics of the feed input. Advantageously, the hyperspectral camera will be able to determine moisture content, lipid content, protein content, ash content and/or particle size. Hyperspectral cameras or sensors collect information as a set of ‘images’. Each image represents a narrow wavelength range of the electromagnetic spectrum, also known as a spectral band. The images may be combined to form a three-dimensional (x,y,λ) hyperspectral data cube for processing and analysis, where x and y typically represent two spatial dimensions of the scene, and λ represents the spectral dimension suitably in a range of wavelengths.

At least one pH sensor may be deployed to determine the pH level of the feed input, or after the larvae have left the substrate. Typically, the pH sensor is deployed as an in line sensor but may also perform sampling. This type of sensor is able to measure the amount of alkalinity and acidity in water and other solutions. pH sensors typically comprise a measuring electrode and a reference electrode. Careful control of the pH level is crucial to determine feed safety for the larvae and may serve as a quality control on input as any variance could be damaging to larvae ecosystem

At least one weighing sensor, optionally part of a feed dosing system may be deployed to determine the mass of feed input in general and/or the mass of feed input at a given stage, for example, the mass of food per larval container, or the mass of food left once the larvae have left the substrate. Monitoring the mass balance and adjusting it, if necessary, throughout the breeding cycle and production system is crucial in order to maximize protein output. In order to monitor the mass balance throughout the fly breeding cycle in line scales and/or a batch monitoring system may be provided. The egg mass produced may be monitored. For example, the feed is dosed via a controlled transportation device such as a screw conveyor into a container on a weigh sensor, once the mass of the feed reaches the required amount the feed is stopped and the container moves on. The weighing sensor may be a standard load cell typically resistive or capacitive.

At least one machine vision system or camera to assess the developmental stage of the eggs, larvae or pupae at a given time by size and/or colour and or chemical composition measurements. The machine vision system of camera may be of the same configuration as described above for fly counting, fly sexing and fly behavioural and health analysis. This advantageously provides insights about egg, larva and pupa health and viability.

At least one larvae counting/egg hatching counting device as described in WO2019/053456A1. Knowing the number of larvae entering the system provides another measure of the state of the apparatus.

At least one gas sensor which may be optionally part of a larger climate control system comprising temperature and humidity sensors as described hereinabove (preferably in line sensors in a climate control system). In embodiments, and advantageously, gas output may be measured in the production and breeding areas of the system. Gas measurements may advantageously provide insights about egg health and viability. Any suitable gas may be monitored by the gas sensor. Oxygen, ammonia, volatile organic compounds (VOCs) and/or carbon dioxide levels may be monitored by the gas sensor.

By way of further explanation, monitoring of gases exhaust in the larval growth units to measure the contents is required to ensure that the larval conditions remain acceptable and to highlight any changes as they occur. Some of the important gases, e.g. Ammonia or VOC's (Volatile Organic Compounds) are highly reactive and therefore difficult to measure or can cause damage to the sensors that detect them, increasing wear rate. Therefore, by assigning proxy gases that are emitted in known relationship with the volatile gases by understanding the biology of the larvae the requirement to measure the gas is made significantly simpler. In other words the level of gas is determined indirectly through its correlation to another less corrosive gas. For example, monitoring of ammonia in production system has a high wear rate on sensor technology, by monitoring a less volatile gas, such as carbon dioxide and extrapolating the amount of ammonia in the air theoretically or via intermittent sampling of the airflow within the production chambers can be optimised.

At least one feedback sensor may be disposed in all processing machinery to provide machine diagnostics in all positions for example to alert maintenance requirements, spare part requirements and product stock status. By way of further explanation, most machines comprise a diagnostic measurement facility of example alerting the user that it is not working or not working correctly, or if maintenance is required. Feedbacks from different machines may be adapted for use with the overall system.

Positional data of mechanical process elements within the breeding system, e.g. pallet of containers or shelves, within the system may be obtained by ceiling mounted cameras, handheld scanners or other capture methods to check barcode, QR code or April tags and identify all elements within the system.

Diet Formulation

Further to the set of inputs outlined above that present in the apparatus, in embodiments there are other inputs external to the apparatus.

One such input is created by an automated software analysis of input and/or feedstock streams available within a given geographical area by their chemical, nutritional and physical properties to create ideal recipes for producing a certain type of larvae. It is known that changes in the diet have a direct causal relationship with the constituents of the larvae once they are processed. Therefore, customer needs can be specifically tailored to and product value optimised by ensuring that the appropriate feedstock streams are sourced in appropriate quantities. Seasonality of given feedstock streams can also be accounted for and predicted within the system.

In embodiments, the diet of the larvae can be monitored via sampling in line and in a laboratory but it is also required that the data produced creates appropriate actions. In embodiments, the control system may comprise diet formulation software for analysing the outputs within the breeding and production systems to tailor specific diets to each area and with respect to the feed streams available within the geographic location of the facility in use it provides the optimum diet and the conditions that diet requires to be most efficiently digested by the larvae. Alternatively, the diet formulation software may be operated distinctly from the control system before or during operation of the apparatus to inform and guide a decision on what the input feed should be.

A high quantity of fruit and vegetables has been shown in combination with other diets to produce high quality larvae, but the cellular structure of these constituents retain moisture longer than other feedstocks, therefore the diet formulation software will provide the requirement to the pre-processing line that this diet will need to be processed longer and broken down to a smaller particle size than a diet with a lower proportion of fruit and vegetable.

In embodiments, the diet formulation software may also take into account customer product needs and specifically tailors the best value output product optimised by ensuring that the appropriate feed stock streams are sourced in appropriate quantities. Seasonality of given feed stocks are also factors accounted for and predicted within the system.

In embodiments, the diet formulation software may be incorporated into the control system.

Output Devices/Control Elements

It is desirable to have the ability to control environmental and physical conditions for the flies at their varying developmental stages within a fly breeding apparatus. As described above, given environmental conditions relating to inter alia, temperature, humidity, gas levels, fly population numbers, sex distribution, health and behaviour can have an influence on the fly population within, however, knowing the state is only one aspect, and control of the same conditions is required to maintain or modulate those conditions in order to achieve a desired outcome.

Any number and type of output devices are envisaged within the apparatus. Suitably, these output devices are intended to control or modify one or more of the conditions of the inputs outlined above. In embodiments, these may include heating or cooling means such as heating pads, or air conditioning chiller units, humidity regulators, gas exchange devices or filters to remove undesired gases, or light controls.

Further specific output devices are described below.

Using Lights to Move Flies and Control Fly Behaviour

Application of specific lighting directions at specific wavelengths can modify the behaviour of flies, for example black soldier flies. By applying these elements at times during the breeding cycle extrapolated from the fly behavioural analysis system by the plant operating system can balance the yield of eggs produced over the lifetime of a cage, flattening the peak and better utilising the egg laying substrates within the cage to reduce overall labour times. It can also be used to direct the flies during movement between the pupation chamber and breeding chamber to increase flow rate and to reduce risk of damage to the flies when the door closes.

It has been shown by others (WO2017072715A1) that artificial lighting can be used for the breeding of black soldier flies.

With the ability to monitor fly behaviour, in particular black soldier fly behaviour, within the breeding chamber it has been shown that there are activities of the black soldier flies that reduce the eggs laid per female fly. In order to counteract the behaviour of black soldier flies that is detrimental to the overall efficiency of the system real time modifications to the lighting within the breeding chamber can be used.

It has been experimentally observed that changing light intensity and wavelength can be used to attract flies, for example, black soldier flies. Therefore, when the distribution of flies within a breeding chamber is not optimum, for example, as measured by the fly behaviour analysis machine described hereinabove, then, in embodiments, the lighting within the chamber may be changed to directly redistribute the flies within the chamber. Ensuring an optimum distribution of flies and a consistent egg yield of the fly population. For example, red light, for example, light of at least about 650 nm and at most about 780 nm, optionally having a light intensity of about 500 lux does not attract a large number of flies but will allow the machine vision system to view the flies. A substantially blue light, for example, light of at least about 420 nm and at most about 520 nm, optionally with an intensity of about 400 lux can be used to attract flies. Substantially blue- and red-light sources may be used alone or in combination.

The ability of light to direct flies by attraction can be used to move fly populations from any part of the apparatus to another.

Changing Light Cycles in the Egg-Growth Chamber to Create Preferred Fall Patterns

One of the components of measurement within the breeding cycle provided in a previous patent (WO2019053456A1) is the number of hatched larvae, by understanding this point of the cycle the numbers of eggs hatching in the system operations upstream and downstream can be changed to ensure the supply is consistent.

One of the automated actions that can ensure a smooth supply across multiple infant larvae counting machine vision systems is to alter the light levels with the system to change the hatch rate of the larvae. It has been demonstrated that varying the light levels can cause greater activity of larvae hatching and then exiting the egg laying substrate to enter the counting system.

If a consistent fly population and consistent egg yield can be maintained or achieved via the means already described above, the variation in numbers of hatching infant larvae is small.

Nevertheless, it has been shown experimentally that infant larvae are more likely to exit the hatching substrate they are stored in if there is an increase in light intensity, for example a difference of more than 50 lux of white light. Other spectrums of light are likely to have the same effect. It has also been observed that infant larvae are less likely to hatch if they are exposed to a light environment over a dark environment. The dark environment may be defined as an environment with less than about 50 lux of light intensity, suitably, 25 lux of light intensity and typically less than about 2 lux of light intensity. These observations ensure that the predominant preferable lighting for the infant larvae to hatch is darkness.

There are natural cycles that mean the hatch rate of infant larvae across a given population varies over time. These natural cycles are accounted for with a large enough population of eggs at different stages of hatching as is seen in a standard facility. Although this is useful to ensure over a period in excess of 24 hours the variation is minor it does not assist with the variation within a period less than 24 hours. Within this time period there can be significant statistical variation of output that results in inconsistent labour requirements to service the output of the equipment.

By switching the lighting on within the larvae hatching chamber as a response when the number drops below a target value of larvae exiting the hatching substrate the supply of larvae out of the larvae hatching area can be kept at a more consistent rate.

Automated Guided Vehicles

Vehicles or other forms of robotic control of movement of physical components within the apparatus may be employed to achieve full automated and/or remote control of operations.

Automated Guided Vehicles may be employed to move batches and/or trays of eggs, larvae or pupae between modules or part of the apparatus, or to other areas within the or each module where environmental conditions are preferable, all under the control system of the system of the present invention.

Control System

The control system of the present invention receives data or inputs as herein defined from one or more of the input devices, as hereinbefore described, and evaluates that data before sending instructions or outputs as herein defined to the appropriate one or more output devices in the apparatus, as hereinbefore described.

The responses of the control system may be determined by a human operator. However, this has certain disadvantages in terms of labour costs and availability, particularly in remote or rural environments.

In embodiments, the responses of the control system are based on, or enhanced, or improved in terms of accuracy and reproducibility of results through an optimisation mechanism utilising some form of automation. Such automation may evaluate the data and send instructions to the one or more outputs without any, or with only minimal, human intervention.

Such automated control systems may make use of pre-programmed or adapted responses to known inputs. Such responses may be based on machine learning algorithms Suitably, the control system uses a neural network, or an alternative machine learning tool, previously trained to recognise the state of the fly population based on input data to determine and send output instructions to the one or more outputs to maintain or achieve a desired condition in the apparatus.

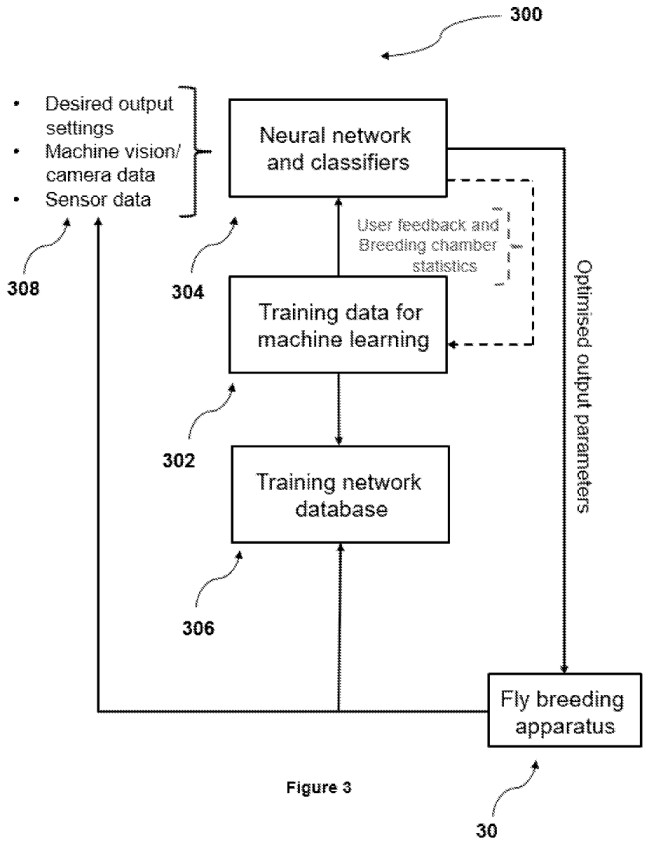

FIG. 3 shows an embodiment of a basic workflow for a machine learning platform 300 that may be used to control the apparatus for breeding flies in accordance with an embodiment of the invention.

Suitably, the system is based on a neural network 304, suitably a convolutional neural network or some other form of artificial neural network, which acts as a classifier, defined as a device that utilizes some training data to understand how given input variables relate to a predetermined class.

In embodiments, the neural network 304, or similar, is primarily trained using training data based on pre-existing data 306 that captures imaging data or other data determining the status of the fly breeding apparatus, and the outcome of various perturbations of the control systems. In embodiments, the training data will be periodically or constantly updated with user feedback (not shown), for example of a trained professional, and fly breeding statistics from ongoing runs of the breeding process to further refine the system. Training data may be image sets taken from the machine vision system that have been analysed by persons skilled in entomology to check for count of flies, sex of flies, health of flies

Once trained the neural network 300 may be presented with data relating to the state of a fly population. In embodiments the data 308 is typically grouped into imaging data obtained by a machine vision system or a camera system, such as shown in FIG. 3 below; and sensor data, such as data from the sensors described below. For example, the sensors may be gas sensors, humidity sensors, pH sensors or temperature sensors or combinations thereof. The neural network may also be provided with desired output settings. Individual types of data may be assigned a certain weighing appropriate to the importance or deemed importance of that data type.

Network Architecture

To extract data from the large number and variety of sensors a network may be used. The network may be a wired and/or wireless (e.g. cellular or wireless LAN) network for transmitting signals to and/or receiving signals between member devices such as an Internet of Things (IoT) network, or a proprietary system bus architecture. The network may allow or Device-to-Device (D2D) communication in which is defined as direct communication between two mobile users without traversing the Base Station (BS) or core network. The D2D network may be combined with another network such as an IoT network or a network based on proprietary system bus connectivity which would allow for network, such as IoT network, compatibility and be a cost effective and operationally simple means of networking non-bespoke machinery throughout the facility. The network may be local, or it may be extended to a remote server away from the apparatus, for example, in the ‘cloud’.

This network allows the agnostic application of all sensors throughout the facility and provides the data securely and remotely so that analysis and action can be taken without geographical limitation. The network, suitably an IoT network or proprietary system bus network, is linked to operator activities and product scanning systems to create a complete picture of the facility through the data and allows for simultaneous monitoring of the environmental and process lines at both micro and macro levels.

A non-exhaustive list of sensory and other data that will be provided to the network, suitably an IoT network or proprietary system bus network, is given below.
Numbers of infant larvae hatched (via machine vision system—see patent WO 2019053456 A1 (Apparatus and methods for production of dipteran insects);
Environmental data within the fly, suitably, black soldier fly, egg hatching chamber (localised within the hatching chamber, measuring humidity and temperature);
Feed input nutritional characteristics (either via sampling and data recording or hyperspectral camera in line monitoring);
Feed input moisture content (in line sensors);
Feed input pH level (in line sensors and/or sampling);
Mass of feed per larval container (Feedback from feed dosing system);
Developmental stage of larvae at a given time, measured via size and/or colour and/or chemical composition (data from sampling via machine vision system);
Temperature of overall breeding area and localised to breeding containers or shelves (Climate control system measurements of temperature and humidity, local measurements using thermal camera systems to measure temperature);
Gas outputs at a macro level in production and breeding areas of the system (in line sensors in climate control systems);
Moisture content of breeding substrate overtime (in line sensors and/or sample analysis)
Frass moisture content after pupae separation (sample analysis);
Frass nutritional content after pupae separation (sample analysis);
Larval nutritional composition output from production processing—fat content, digestibility, protein quality, water content. (sample analysis);
Mass balance throughout the breeding cycle and production system (in line scales and batch monitoring system);
Emergence rate of flies from pupae (fly counting system);
Pupa developmental stage—via colour or activity level (data from sampling via machine vision system);
Number of flies per breeding chamber (fly counting system);
Total number of flies in the system (fly counting system);
Sex, behaviour and health of flies (fly sex, behaviour and health monitoring system)
Egg mass produced (in line scales);
Egg health and viability measured via machine vision sample analysis (data from sampling via machine vision system and/or gas analysis);
Positional data of mechanical process elements, e.g. pallet of containers or shelves, within the facility (ceiling mounted cameras, handheld scanners or other capture method to check barcode, QR code or April tags and identify all elements within the system);
Machine diagnostics in all positions (feedback sensors in processing machinery);
Maintenance requirements for machines (feedback sensors in processing machinery and input data on maintenance requirements);
Spare part requirements (direct input from operators);
Product stock status (batch processing data, barcode or QR code capture).

All of these inputs into the network, suitably an IoT network or proprietary system bus network, will come directly from sensors or input by operators as part of the system as herein described. This information is centrally processed, suitably by the control system as herein described and will either report status to an operator and/or will create direct actions to outputs, such as those herein described maintain the integrity of the process. A non-exhaustive list of direct output actions from the network, suitably an IoT network or proprietary system bus network, is listed below.
Alterations in environment or lighting within infant larvae hatching unit to smooth hatch rate of larvae and ensure homeostatic infant population
Variation of feed input moisture content or nutritional constitution (can change feed input recipe and the machine operation time for dewatering to change moisture content)
Frequency of feeding of breeding larvae stock (feeding larvae on day 9 instead of day 10 will have an effect on feed substrate drying time and larval development time)
Density of larvae per breeding container (more larvae per breeding container means smaller larvae but more efficient use of feed)
Density of larvae per production container (more larvae per breeding container means smaller larvae but more efficient use of feed)
Environmental changes for breeding system, changing humidity, temperature or airflow through the breeding containers or shelves (changes to macro climate conditions throughout facility or localised air flow changes or batch positions related to climate systems)
Moisture content may be changed via changes to feedstock input or via filtration or mechanical action methods within the breeding containers or shelves
Frequency of sampling of larvae batches for quality analysis (greater variation in results may require more extensive sampling, the central processor will be able to change sampling frequency to provide more data to identify issues)
Mixing requirement or frequency if required to mix substrate in breeding or production batches (breeding larvae may need to be mixed to ensure a lack of mould build up, this frequency depends on the feedstock and environment)
Lifespan and frequency of operations of pupae storage chambers population (pupae storage chambers hold a pupae population until they become flies and are periodically released, the amount of time between releases)
Lighting requirements in breeding chambers (Distribution of lighting affecting the behaviour of the black soldier flies)
Egg collection frequency within breeding chambers (mass of eggs removed from breeding chambers will determine the frequency of removals required)
Aperture size between pupae storage chamber and breeding chamber during release of flies (When the flow rate of the flies into the breeding chamber needs to be reduced)
Aperture closing between pupae storage chamber and breeding chamber during release of flies (When the flow rate of the flies into the breeding chamber needs to be stopped)
Lifespan of breeding chambers and quality of cleaning required after process (knowing the number of live flies in the breeding chamber will determine when it is efficient to end its operation cycle and the machine vision system can determine when a higher cleaning regime is required to improve the background quality)

The data from the network, suitably an IoT network or proprietary system bus network, may be analysed within the operating system of the control system. A virtual facility is thereby created with idealised optimal boundaries and efficiencies throughout the system. This is fed with real input and output data and is iterated to identify the levers within the system that create systemic changes. The virtual facility can then be modified by external operators to optimise the process efficiency via incremental iterative testing, this is then tried in the real facility and the system feeds back the results. This allows the system to be tested and continually improved.

The virtual facility will provide boundaries to all of the environmental and process variables in the real facility and the plant operating system will ensure that the real facility acts to stay within those boundaries. It is especially important that across the complex biological system that is the fly, in particular, Black Soldier Fly, rearing and production stages the whole system is considered simultaneously and as a whole, which is only possible by bringing all of the data into a single place and a single operating system.

### EXAMPLES

### Example 1: Comparison of Fly Counting Using a Machine Vision System in Accordance with the Present Invention and Manual Fly Counting

Objective:

To provide an automated system to identify and count the number of flies in a breeding chamber.

Method:

Seven 20 Megapixel CMOS area scan cameras were mounted in a fly chamber, six on the walls of the chamber, one in the ceiling. This provided view planes covering all and each of the internal walls and the floor of the chamber. The ceiling of the breeding chamber was excluded from the count due to the very low number of flies present on it and the difficulty in keeping a floor mounted camera clean.

Overlapping regions of the images captured by the cameras were minimised by suitable positioning and adjustment of each camera angle. Regions of the walls and floor that remain overlapped in the captured images were identified and flies in this region were counted using only one camera image of the area to avoid double counting of flies using software techniques.

Any obstructing elements within the fly chamber (egg laying substrates, water provision etc should not be used during fly counter trials) were minimised.

The walls, floor and ceiling of the breeding chamber had a while background colour. In this example, directed jets of air were passed over each camera lens to ensure the camera view is not obscured by settling flies.

Flies recently emerged from pupae were released into the breeding chamber to provide the approximate target of 25,000 flies in the chamber.

Images were taken of flies in the breeding chamber and the number of flies present were calculated based on extrapolated data of the number of flies imaged by each camera on the viewed wall or floor. While in principle the cameras may be directed to any view plane, range of focus, or volume within the breeding chamber, in this example, the cameras detected flies on each wall and the floor.

While the number of flies may be counted from one captured image set, in this example multiple image sets (400-800) were captured with a time interval between sets of approximately 10 seconds and the results compared or averaged (number mean average, or other suitable average). This technique may be useful to increase accuracy or reduce noise for counting of a fixed population enclosure, or over a time period in an enclosure when the population can be deemed to be constant or varying only minimally. In this way, while the individual flies captured in each image vary, the fly count in a fixed population enclosure should remain the same meaning errors in counting are reduced. For an increasing fly population, for example when the flies are being released into a chamber, a moving average may be used over a calculated time interval to provide a count less susceptible to errors. The same or similar technique may be applied for any imaging process described herein, for example, fly counting, fly sexing, determining fly health, and/or determining fly behaviour.

Captured image data was analysed using machine vision detection methods (blob or shape analysis), combining them to identify a fly. Each fly identified in an image is then assigned a number and the total is counted.

Collect flies and place in freezer for two days to deactivate any pests or any eggs that may have been laid on the flies.

Manually count all flies from the breeding chamber and compare the manual count number achieved to fly counter data.

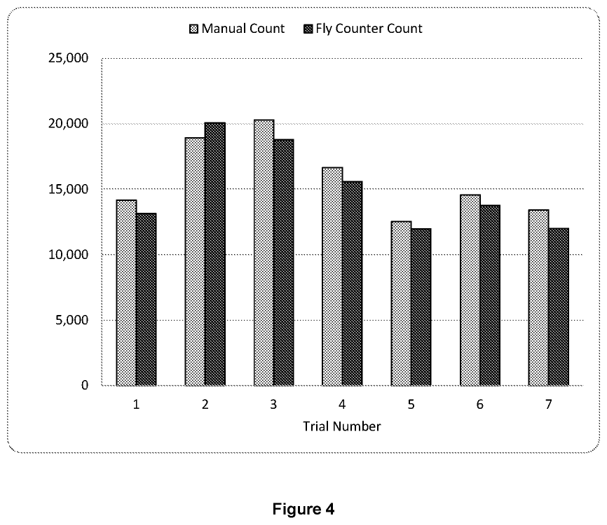

The results of the comparison tests are provided in Table 1 below, and pictorially in FIG. 4.

TABLE 1

| | Full Manual | Fly Counter | Percentage |
|---|---|---|---|
| Trial | Count | Count | difference |
| 1 | 14,165 | 13145 | 93% |
| 2 | 18,920 | 20046 | 106% |
| 3 | 20,262 | 18766 | 93% |
| 4 | 16,639 | 15564 | 94% |
| 5 | 12,526 | 11,954 | 95% |
| 6 | 14,564 | 13,763 | 95% |
| 7 | 13,418 | 11,994 | 89% |

The results show a good correlation (within 10%) of machine vision system count with the manual counting control figure. Key to this data is also that it is often lower by a similar amount, which would allow the output figure to be slightly compensated to give a more accurate value.

While these results offer a significant improvement in the ability to monitor fly populations in real time, or at least in much reduced time compared to other known methods such as manual counting, with further development of the system, it would be anticipated that even closer correlation of the automated count to the manual count may be achieved.

### Example 2: Fully Automated Fly Counting

The system above may be further improved by the use of machine learning techniques, trained using based on the manual counting data received compared to the automated count from the machine vision system. This may be beneficial in the early stages of development, and as part of an ongoing maintenance of the system with any discrepancy between the actual number of flies used to further train algorithm for repeat counts.

Although particular embodiments of the invention have been disclosed herein in detail, this has been done by way of example and for the purposes of illustration only. The aforementioned embodiments are not intended to be limiting with respect to the scope of the invention. It is contemplated by the inventor that various substitutions, alterations, and modifications may be made to the invention without departing from the scope of the invention.

1. A system for controlling a fly breeding apparatus, wherein the fly breeding apparatus comprises one or more enclosures for the containment of a population of flies;
wherein the system comprises:
one or more input devices, wherein at least one of the one or more input devices is a machine vision system or camera that is configured for imaging the population of flies, or a portion thereof, within at least one of the one or more enclosures;
one or more output devices; and
a control system;
wherein, the one or more input devices, the one or more output devices and the control system are connected to enable the system to control and/or maintain at least one property of a status of the population of flies within the fly breeding apparatus.

2. The system of claim 1, wherein the at least one property of a status of the population of flies is selected from the group consisting of: a total number of flies in the population of flies; a total number of female flies in the population of flies; a total number of male flies in the population of flies; a ratio of the number of female flies to male flies in the population of flies; the health of the population of flies; the behaviour of the population of flies; and combinations thereof.

3. The system of claim 1 or claim 2, wherein the one or more input devices are selected from the group consisting of: a further machine vision system or a camera; a hyperspectral camera; a gas sensor; a temperature sensor; a pH sensor; a humidity sensor; a weight sensor; a feedback sensor; and combinations thereof.

4. The system of any one of claims 1 to 3, wherein the one or more output devices are selected from the group consisting of: lights; feed input controls; larvae input controls; humidifiers; dehumidifiers; heaters; refrigeration or cooling means; gas control valves; mass gas flow devices; motors in automated guided vehicles.

5. The system of any one of claims 1 to 4, wherein the one or more output devices are able to control one or more condition within the fly breeding apparatus, selected from the group consisting of: lighting within the fly breeding apparatus or parts thereof; amount of feed input; moisture content of feed input; nutritional constitution of feed input; frequency of feeding; density of larvae; humidity; temperature; gas concentration; airflow through the breeding apparatus or parts thereof; and control of automated guided vehicles within the fly breeding apparatus.

6. The system of any one of claims 1 to 5, wherein the control system is configured to:
a) receive one or more inputs from the or each of the one or more of the input devices;
b) evaluate the one or more inputs; and
c) send one or more outputs to the or each of the one or more output devices.

7. The system of any one of claims 1 to 6, wherein the control system is an autonomous optimisation mechanism utilising machine learning.

8. The system of claim 7, wherein, the control system comprises one of more machine learning techniques selected from the group consisting of: a neural network; machine learning models; or a combination thereof.

9. The system of any one of claims 1 to 8, wherein the system further comprises one or more interfaces between the control system and a wired and/or a wireless network for transmitting signals to and/or receiving signals from a local or a remote location.

10. The system of claim 9, wherein the control system is configured to transmit data to, and/or receive data from a remote location.

11. A network of connected devices for controlling a fly breeding apparatus, wherein the fly breeding apparatus comprises one or more enclosures for the containment of a population of flies;
wherein the network comprises:
one or more input devices, wherein at least one of the one or more input devices is a machine vision system or camera that is configured for imaging the population of flies, or a portion thereof, within at least one of the one or more enclosures;
one or more output devices; and
a control system,
wherein, the network controls and/or maintains at least one property of a status of a population of flies within the fly breeding apparatus.

12. The network of claim 11, wherein the at least one property of a status of the population of flies is selected from the group consisting of: a total number of flies in the population of flies; a total number of female flies in the population of flies; a total number of male flies in the population of flies; a ratio of the number of female flies to male flies in the population of flies; the health of the population of flies; the behaviour of the population of flies; and combinations thereof.

13. The network of claim 11 or claim 12, wherein the one or more inputs are selected from the group consisting of: a further machine vision system or a camera; a hyperspectral camera; a gas sensor; a temperature sensor; a pH sensor; a humidity sensor; a weight sensor; a feedback sensor; and combinations thereof.

14. The network of any one of claims 11 to 13, wherein the one or more output devices are selected from the group consisting of: lights; feed input controls; larvae input controls; humidifiers; dehumidifiers; heaters; refrigeration or cooling means; gas control valves; mass gas flow devices; motors in automated guided vehicles.

15. The network of any one of claims 11 to 14, wherein the one or more output devices are able to control one or more condition within the fly breeding apparatus, selected from the group consisting of: lighting within the fly breeding apparatus or parts thereof; amount of feed input; moisture content of feed input; nutritional constitution of feed input; frequency of feeding; density of larvae; humidity; temperature; gas concentration; airflow through the breeding apparatus or parts thereof; and control of automated guided vehicles within the fly breeding apparatus.

16. The network of any one of claims 11 to 15, wherein the control system is configured to:
a) receive one or more inputs from the or each of the one or more of the input devices;
b) evaluate the one or more inputs; and
c) send one or more outputs to the or each of the one or more output devices.

17. The network of any one of claims 11 to 16, wherein the network comprises a wired and/or wireless connection between the one or more input devices, the one or more output devices and the control system.

18. The network of any one of claims 11 to 17, wherein the network is used in the system of any one of claims 1 to 10.

19. A method for controlling a fly breeding apparatus, wherein the fly breeding apparatus comprises one or more enclosures for the containment of a population of flies;
wherein the method comprises:
a) Providing a fly breeding apparatus;
b) Providing a system for controlling a fly breeding apparatus, wherein the system comprises:
i. one or more input devices, wherein at least one of the one or more input devices is a machine vision system or camera that is configured for imaging the population of flies, or a portion thereof, within at least one of the one or more enclosures;
ii. one or more output devices; and
iii. a control system;
c) The control system receives inputs from the or each of the one or more input devices;
d) The control system evaluates the inputs from the or each of the one or more input devices;
e) The control system provides outputs to the one or more output devices;
f) The one or more output devices respond to the outputs to control and/or maintain at least one property of a status of a population of flies within the fly breeding apparatus.

20. The method of claim 19, wherein the at least one property is a total number of flies in the population of flies; a total number of female flies in the population of flies; a total number of male flies in the population of flies; a ratio of the number of female flies to male flies in the population of flies; the health of the population of flies; the behaviour of the population of flies; and combinations thereof.

21. The method of claim 19 or claim 20, wherein the one or more input devices are selected from the group consisting of: a further machine vision system or a camera; a hyperspectral camera; a gas sensor; a temperature sensor; a pH sensor; a humidity sensor; a weight sensor; a feedback sensor; and combinations thereof.

22. The method of any one of claims 19 to 21, wherein the one or more output devices are selected from the group consisting of: lights; feed input controls; larvae input controls; humidifiers; dehumidifiers; heaters; refrigeration or cooling means; gas control valves; mass gas flow devices; motors in automated guided vehicles.

23. The method of any one of claims 19 to 22, wherein the one or more output devices are able to control one or more condition within the fly breeding apparatus, selected from the group consisting of: lighting within the fly breeding apparatus or parts thereof; amount of feed input; moisture content of feed input; nutritional constitution of feed input; frequency of feeding; density of larvae; humidity; temperature; gas concentration; airflow through the breeding apparatus or parts thereof; and control of automated guided vehicles within the fly breeding apparatus.

24. The system of any one of claims 1 to 10, the network of any one of claims 11 to 18 and the method of any one of claims 19 to 23, wherein the machine vision system comprises at least one camera.

25. The system, network, or method of claim 24, wherein the or each camera has a resolution of greater than 5 megapixels.

26. The system of any one of claims 1 to 10, 24 and 25, the network of any one of claims 11 to 18, 24 and 25, and the method of any one of claims 19 to 25, wherein the machine vision system is configured to image flies on one of more of: an interior surface of the enclosure or part thereof; an interior volume of the enclosure or part thereof; a plane bisecting the interior volume of the enclosure or part thereof; and combinations thereof.

27. The system of any one of claims 1 to 10 and 24 to 26, the network of any one of claims 11 to 18 and 24 to 26, and the method of any one of claims 19 to 26, wherein the machine vision system is configured to detect the number of flies; the sex of flies; the health status of flies; and/or the behaviour status of flies.

28. A machine vision system for determining at least one property of a status of a population of flies within a fly breeding apparatus, wherein the machine vision system comprises:
a) an enclosure for the containment of a population of flies;
b) one or more image capture devices aimed inwardly into the interior of the enclosure.

29. The machine vision system of claim 28, wherein the machine vision system comprises at least one camera.

30. The machine vision system of claim 28 or claim 29, wherein the or each camera has a resolution of greater than 5 megapixels.

31. The machine vision system of any one of claims 28 to 30, wherein the machine vision system is configured to image flies on one of more of: an interior surface of the enclosure or part thereof; an interior volume of the enclosure or part thereof; a plane bisecting the interior volume of the enclosure or part thereof; and combinations thereof.

32. The machine vision system of any one of claims 28 to 31, wherein the machine vision system is configured to detect the number of flies; the sex of flies; the health status of flies; and/or the behaviour status of flies.

33. A method of counting flies using the system of any one of claims 1 to 10, the network of any one of claims 11 to 18, or the machine vision system of any one of claims 28 to 32.

34. A method of determining the ratio of male and female flies using the system of any one of claims 1 to 10, the network of any one of claims 11 to 18, or the machine vision system of any one of claims 28 to 32.

35. A method of determining the health status of flies using the system of any one of claims 1 to 10, the network of any one of claims 11 to 18, or the machine vision system of any one of claims 28 to 32.

36. A method of determining the behaviour status of flies using the system of any one of claims 1 to 10, the network of any one of claims 11 to 18, or the machine vision system of any one of claims 28 to 32.

37. The method of any one of claims 33 to 36, wherein the method is based on extrapolation of a result from a sample area or volume, wherein the sample area or volume is less than or smaller than the area or volume of the whole area or volume, or a defined part thereof.

38. The method of claim 37, wherein extrapolation is based on applying a multiplier to the result from sample area or volume based on of the ratio of the sample area or volume to the whole area or volume.

39. The method of claim 38, wherein the multiplier is a simple or weighted multiplier.

40. The method of claim 39, wherein the weighting of the weighted amplifier is based on the anticipated or known variations in fly numbers on different surfaces on volumes compared with the sample area or volume imaged.

41. A fly breeding apparatus comprising the system of any one of claims 1 to 10, the network of any one of claims 11 to 18 and/or the machine vision system of any one of claims 28 to 32.
